(19) United States
(12) Patent Application Publication
(10) Pub. No.: US 2014/0257489 A1
(43) Pub. Date: Sep. 11, 2014

(54) METHOD AND APPARATUS FOR MINIMALLY INVASIVE INSERTION OF INTERVERTEBRAL IMPLANTS

(71) Applicant: Interventional Spine, Inc., Irvine, CA (US)

(72) Inventors: Christopher R. Warren, Trabuco Canyon, CA (US); Robert J. Flower, Sun City, CA (US); Fausto Olmos, Laguna Niguel, CA (US)

(21) Appl. No.: 13/827,531

(22) Filed: Mar. 14, 2013

Related U.S. Application Data
(60) Provisional application No. 61/849,822, filed on Mar. 11, 2013.

(51) Int. Cl. A61M 29/00; A61F 2/44

(52) U.S. Cl. CPC A61M 29/00; A61F 2/442
USPC 623/17.16; 606/86 A

(57) ABSTRACT

A dilation introducer for orthopedic surgery is provided for minimally invasive access for insertion of an intervertebral implant. The dilation introducer may be used to provide an access position through Kambin's triangle from a posterolateral approach. A first dilator tube with a first longitudinal axis is provided. A second dilator tube may be introduced over the first, advanced along a second longitudinal axis parallel to but offset from the first. A third dilator tube may be introduced over the second, advanced along a third longitudinal axis parallel to but offset from both the first and the second. An access cannula may be introduced over the third dilator tube. With the first, second, and third dilator tubes removed, surgical instruments may pass through the access cannula to operate on an intervertebral disc and/or insert an intervertebral implant. The access cannula may have a substantially rectangular cross-section.

### BACKGROUND OF THE INVENTION

1. Field of the Invention

The present application relates to medical devices and, more particularly, to a medical device and method for treating the spine.

2. Description of the Related Art

The human spine is a flexible weight bearing column formed from a plurality of bones called vertebrae. There are thirty-three vertebrae, which can be grouped into one of five regions (cervical, thoracic, lumbar, sacral, and coccygeal). Moving down the spine, there are generally seven cervical vertebrae, twelve thoracic vertebrae, five lumbar vertebrae, five sacral vertebrae, and four coccygeal vertebrae. The vertebrae of the cervical, thoracic, and lumbar regions of the spine are typically separate throughout the life of an individual. In contrast, the vertebra of the sacral and coccygeal regions in an adult are fused to form two bones, the five sacral vertebrae which form the sacrum and the four coccygeal vertebrae which form the coccyx.

In general, each vertebra contains an anterior, solid segment or body and a posterior segment or arch. The arch is generally formed of two pedicles and two laminae, supporting seven processes—four articular, two transverse, and one spinous. There are exceptions to these general characteristics of a vertebra. For example, the first cervical vertebra (atlas vertebra) has neither a body nor spinous process. In addition, the second cervical vertebra (axis vertebra) has an odontoid process, which is a strong, prominent process, shaped like a tooth, rising perpendicularly from the upper surface of the body of the axis vertebra. Further details regarding the construction of the spine may be found in such common references as Gray's Anatomy, Crown Publishers, Inc., 1977, pp. 33-54, which is herein incorporated by reference.

The human vertebrae and associated connective elements are subjected to a variety of diseases and conditions which cause pain and disability. Among these diseases and conditions are spondylosis, spondylolisthesis, vertebral instability, spinal stenosis and degenerated, herniated, or degenerated and herniated intervertebral discs. Additionally, the vertebrae and associated connective elements are subject to injuries, including fractures and torn ligaments and surgical manipulations, including laminectomies.

The pain and disability related to the diseases and conditions often result from the displacement of all or part of a vertebra from the remainder of the vertebral column. Over the past two decades, a variety of methods have been developed to restore the displaced vertebra to their normal position and to fix them within the vertebral column. Spinal fusion is one such method. In spinal fusion, one or more of the vertebra of the spine are united together (“fused”) so that motion no longer occurs between them. Thus, spinal fusion is the process by which the damaged disc is replaced and the spacing between the vertebrae is restored, thereby eliminating the instability and removing the pressure on neurological elements that cause pain.

Spinal fusion can be accomplished by providing an intervertebral implant between adjacent vertebrae to recreate the natural intervertebral spacing between adjacent vertebrae. Once the implant is inserted into the intervertebral space, osteogenic substances, such as autogenous bone graft or bone allograft, can be strategically implanted adjacent the implant to prompt bone ingrowth in the intervertebral space. The bone ingrowth promotes long-term fixation of the adjacent vertebrae. Various posterior fixation devices (e.g., fixation rods, screws etc.) can also be utilize to provide additional stabilization during the fusion process.

Notwithstanding the variety of efforts in the prior art described above, these intervertebral implants and techniques are associated with another disadvantage. In particular, these techniques typically involve an open surgical procedure, which results in higher cost, lengthy in-patient hospital stays and the pain associated with open procedures. In addition, many intervertebral implants are inserted anteriorly while posterior fixation devices are inserted posteriorly. This results in additional movement of the patient. Therefore, there remains a need in the art for an improved apparatus and method for introducing an intervertebral implant.

### SUMMARY OF THE INVENTION

In one embodiment, the implant is advantageously introduced via a minimally invasive procedure, taking a posterolateral approach at least partially through Kambin's triangle in a manner that advantageously provides protection to the exiting and traversing nerves. In one arrangement, to facilitate introduction of instruments and/or devices at least partially through Kambin's triangle a foraminoplasty is performed. In one embodiment, the foraminoplasty is performed using one or more features provided one or more dilator tubes that can be used to dilate tissue.

In accordance with an embodiment, a dilation introducer for orthopedic surgery comprises: a first dilator tube having a substantially circular cross-section; a second dilator tube having a first longitudinal lumen configured to slidably receive the first dilator therein, wherein the outer surface of the second dilator tube has a substantially rectangular cross-section; and an access cannula having a second longitudinal lumen configured to slidably receive the second dilator therein, wherein the cross-section of the second longitudinal lumen is substantially rectangular. In one embodiment, the access cannula has at least one flat side. In another embodiment, the access cannula has at least two flat sides that can be positioned adjacent to each other or opposing each other. In another embodiment, the access cannula has at least two flat sides that are substantially at right angles to each other. In another embodiment, the access cannula has at least three flat sides that are substantially at right angles to each other.

In some embodiments, the cross-section of the second longitudinal lumen is substantially square. In some embodiments, the second longitudinal lumen has a height and a width of approximately 10 mm. In some embodiments, the cross-section of second longitudinal lumen configured to receive an intervertebral implant therethrough. In some embodiments, wherein the first longitudinal lumen is centered with respect to the outer surface of the second dilator tube. In some embodiments, the access cannula comprises an outer surface having a substantially rectangular cross-section. In some embodiments, distal end of the access cannula is beveled such that a cross-section of the second longitudinal lumen at the distal end of the access cannula is U-shaped. In some embodiments, the dilation introduce is configured for removably connecting the first and second dilator tubes together in a locked arrangement, whereby in the locked arrangement the slidable movement is restricted. In some embodiments, the second dilator tube is rotatable with respect to the first dilator tube around the first longitudinal axis. In some embodiments, the first dilator tube contains cutting flutes on at least one side. In some embodiments, the access cannula has a smooth outer surface.

In accordance with another embodiment, a method for accessing a patient's intervertebral disc to be treated in orthopedic surgery comprises the steps of: passing a first dilator tube along a first longitudinal axis through Kambin's triangle until the first dilator tube reaches the intervertebral disc to be treated; passing a second dilator tube along a second longitudinal axis that is parallel to and laterally displaced from the first longitudinal axis, until the distal end of the second dilator contacts the annulus, wherein the second dilator tube has cutting flutes oriented towards the inferior pedicle, and wherein the distal portion of the second dilator tube has a generally semi-annular cross-section, configured such that the second dilator tube does not contact the exiting nerve during insertion; passing an access cannula over the second dilator tube until the distal end of the access cannula contacts the annulus, wherein the access cannula has an outer surface with a substantially rectangular cross-section.

In some embodiments, the method can further comprise passing a third dilator tube over the second dilator tube along the second longitudinal axis until the distal end of the third dilator contacts the annulus, wherein the distal portion of the third dilator tube is beveled such that the third dilator tube does not contact the exiting nerve during insertion, wherein the access cannula is passed over the third dilator tube. In some embodiments, the method can further comprise forming a further recess in the inferior pedicle by rotating the second dilator tube back and forth. In some embodiments, the method can further comprise forming a further recess in the inferior pedicle by longitudinally sliding the second dilator tube back and forth. In some embodiments, the method can further comprise passing the access cannula over the third dilator tube until the distal end of the third dilator contacts the annulus such that the access cannula does not contact the exiting nerve during insertion; rotating the access cannula such that generally U-shaped cross-section opens opposite the exiting nerve; removing the first, second, and third dilator tubes. In some embodiments, the method can further comprise operating on an intervertebral disc by inserting surgical instruments through the access cannula.

In accordance with another embodiment, a method for performing orthopedic surgery comprises: introducing a first dilator tube through Kambin's triangle; introducing a second dilator tube over the first dilator tube; and introducing an access cannula over the first and second dilator tubes, the access cannula having a substantially rectangular cross-section.

In some embodiments, the method further comprises removing bone from the inferior pedicle with the first dilator tube prior to introducing the access cannula. In some embodiments, the method further comprises operating on the spine through the access cannula.

In accordance with another embodiment, a method for accessing a patient's intervertebral disc to be treated in orthopedic surgery comprises the steps of: performing a foraminoplasty; inserting an access cannula through the enlarged opening created by the foraminoplasty, the access cannula having a substantially rectangular cross-section; and introducing devices or tools into the intervertebral disc through the access cannula.

In some embodiments, the method further comprises introducing an implant into the intervertebral disc. In some embodiments, the method further comprises expanding the implant within the disc. In some embodiments, the foraminoplasty is performed at least partially using cutting surfaces on one or more dilator tubes. In some embodiments, the method further comprises inserting trans-facet screws into a facet joint.

Other features and advantages of the present invention will become more apparent from the following detailed description of the preferred embodiments in conjunction with the accompanying drawings, which illustrate, by way of example, the operation of the invention.

### BRIEF DESCRIPTION OF THE DRAWINGS

The abovementioned and other features of the inventions disclosed herein are described below with reference to the drawings of the preferred embodiments. The illustrated embodiments are intended to illustrate, but not to limit the inventions. The drawings contain the following figures:

FIG. 1 is a lateral elevational view of a portion of a vertebral column.

FIG. 2 is a schematic side view of Kambin's triangle.

FIG. 3 is a perspective view of an access cannula in positioned against a vertebral column.

FIG. 4A is a plan view of an embodiment of a second dilator tube.

FIG. 4B is an enlarged detail view of the distal end of the second dilator tube shown in FIG. 4A.

FIG. 4C is an enlarged detail view of the proximal end of the second dilator tube shown in FIG. 4A.

FIG. 5A is a plan view of an embodiment of a third dilator tube.

FIG. 5B is an enlarged detail view of the distal end of the third dilator tube shown in FIG. 5A.

FIG. 5C is an enlarged detail view of the proximal end of the third dilator tube shown in FIG. 5A.

FIG. 5D is a front view of the third dilator tube shown in FIG. 5A.

FIG. 6A is a side view of an embodiment of an access cannula.

FIG. 6B is an enlarged detail view of the distal end of the access cannula shown in FIG. 6A.

FIG. 6C is an enlarged detail view of the proximal end of the access cannula shown in FIG. 6A.

FIG. 7A is a plan view of an embodiment of a dilation introducer comprising the second dilator tube of FIG. 4A, the third dilator tube of FIG. 5A, and the access cannula of FIG. 6A.

FIG. 7B is an enlarged detail view of the distal end of the dilation introducer shown in FIG. 7A.

FIG. 7C is an enlarged detail view of the proximal end of the dilation introducer shown in FIG. 7A.

FIG. 8A is a longitudinal cross-sectional view of the dilation introducer of FIG. 7A.

FIG. 8B is an enlarged detail of the longitudinal cross-sectional view shown in FIG. 8A.

FIGS. 9A-9C show a method of insertion of a first dilator tube or trocar into the intervertebral space.

FIG. 10A is a perspective view of the dilation introducer of FIG. 7A positioned against the spine.

FIG. 10B is an enlarged detail view of a distal end of the dilation introducer of FIG. 7A.

FIG. 11 is a perspective view of the dilation introducer of FIG. 7A, with the third dilator tube introduced over the second dilator tube.

FIG. 12 shows the access point before and after the foraminoplasty performed by the dilation introducer of FIG. 7A.

FIG. 13A is a perspective view of the dilation introducer of FIG. 7A, with the access cannula introduced over the third dilator tube.

FIG. 13B is a perspective view of the dilation introducer of FIG. 7A, with the access cannula rotated to protect the exiting nerve.

FIG. 13C is a perspective view of the dilation introducer of FIG. 7A, with the first, second, and third dilator tubes removed, while the access cannula remains in place.

FIG. 14 is a plan view of an intervertebral implant for delivery through the access cannula.

FIG. 15A is a perspective view of another embodiment of an intervertebral implant in an unexpanded state.

FIG. 15B is a perspective view of the intervertebral implant shown in FIG. 15A wherein the implant is in an expanded state.

FIG. 16 is a bottom view of the intervertebral implant shown in FIG. 15A.

FIG. 17 is a side view of the intervertebral implant shown in FIG. 15B.

FIG. 18 is a front cross-sectional view of the intervertebral implant shown in FIG. 16B taken along lines 19-19.

FIG. 19A is a bottom perspective view of a lower body portion of the intervertebral implant shown in FIG. 18A.

FIG. 19B is a top perspective view of the lower body portion of the intervertebral implant shown in FIG. 18A.

FIG. 20A is a bottom perspective view of an upper body portion of the intervertebral implant shown in FIG. 18A.

FIG. 20B is a top perspective view of the upper body portion of the intervertebral implant shown in FIG. 18A.

FIG. 21 is a perspective view of an actuator shaft of the intervertebral implant shown in FIG. 15A.

FIG. 22A is a front perspective view of a proximal wedge member of the intervertebral implant shown in FIG. 15A.

FIG. 22B is a rear perspective view of the proximal wedge member of the intervertebral implant shown in FIG. 15A.

FIG. 23A is a front perspective view of a distal wedge member of the intervertebral implant shown in FIG. 15A.

FIG. 23B is a rear perspective view of the distal wedge member of the intervertebral implant shown in FIG. 15A.

FIG. 24 is a perspective view of a deployment tool according to an embodiment.

FIG. 25 is a side cross-sectional view of the deployment tool shown in FIG. 24 wherein an expandable implant is attached to a distal end thereof.

FIG. 26A illustrates a perspective view of another embodiment of a deployment tool.

FIGS. 26B and 26C illustrate an enlarged perspective views of the distal end of the deployment tool of FIG. 26A, with and without an engaged intervertebral implant.

FIG. 27A is a plan view of a plunger assembly for a graft delivery system, according to an embodiment.

FIG. 27B is a longitudinal cross-sectional view of the plunger assembly shown in FIG. 27A.

FIG. 28A is a plan view of a funnel assembly for a graft delivery system, according to an embodiment.

FIG. 28B is a schematic view of the funnel assembly shown in FIG. 28A.

FIG. 28C is an end view of the funnel assembly shown in FIG. 28A.

FIG. 28D is a longitudinal cross-sectional view of the funnel assembly shown in FIG. 28A.

### DETAILED DESCRIPTION OF THE PREFERRED EMBODIMENTS

In accordance with certain embodiments disclosed herein, an improved apparatus for inserting an intervertebral implant is provided. For example, in one embodiment, the apparatus may be used to insert surgical instruments and/or one or more intervertebral implants through a minimally invasive procedure to reduce trauma to the patient and thereby enhance recovery and improve overall results. By minimally invasive, Applicant means a procedure performed percutaneously through an access device in contrast to a typically more invasive open surgical procedure.

Certain embodiments disclosed herein are discussed in the context of an intervertebral implant and spinal fusion because of the device and methods have applicability and usefulness in such a field. The device can be used for fusion, for example, by inserting an intervertebral implant to properly space adjacent vertebrae in situations where a disc has ruptured or otherwise been damaged. “Adjacent” vertebrae can include those vertebrae originally separated only by a disc or those that are separated by intermediate vertebra and discs. Such embodiments can therefore be used to create proper disc height and spinal curvature as required in order to restore normal anatomical locations and distances. However, it is contemplated that the teachings and embodiments disclosed herein can be beneficially implemented in a variety of other operational settings, for spinal surgery and otherwise.

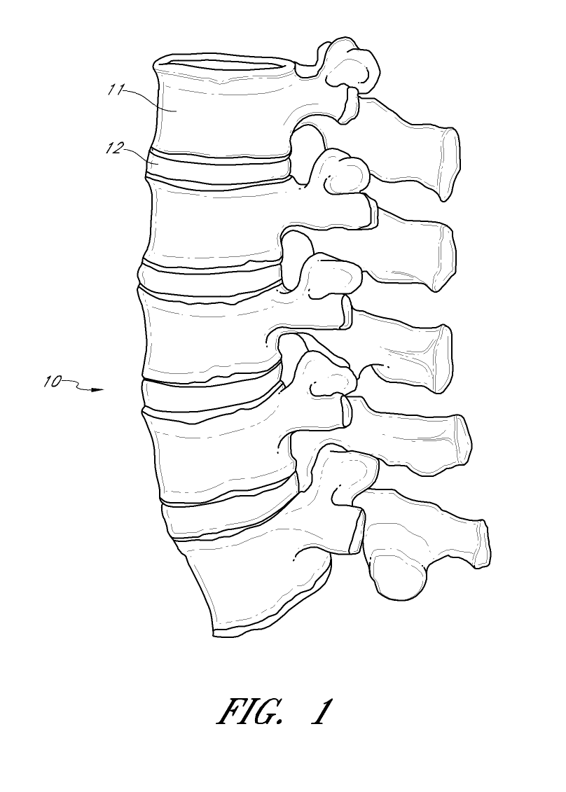

As context for the methods and devices described herein, FIG. 1 is a lateral view of a vertebral column 10. As shown in FIG. 1, the vertebral column 10 comprises a series of alternative vertebrae 11 and fibrous intervertebral discs 12 that provide axial support and movement to the upper portions of the body. The vertebral column 10 typically comprises thirty-three vertebrae 11, with seven certical (C1-C7), twelve thoracic (T1-T12), five lumbar (L1-L5), five fused sacral (S1-S5), and four fused coccygeal vertebrae.

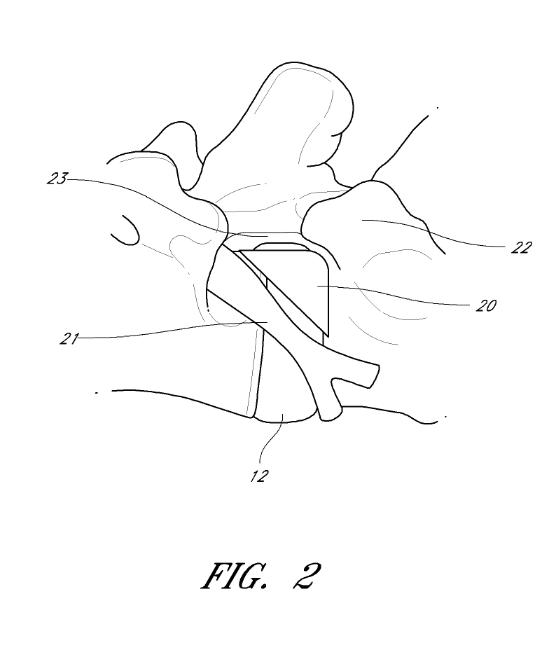

FIG. 2 is a schematic view of Kambin's triangle. This region 20 is the site of posterolateral access for spinal surgery. It can be defined as a right triangle over the intervertebral disc 12 viewed dorsolaterally. The hypotenuse is the exiting nerve 21, the base is the superior border of the inferior vertebra 22, and the height is the traversing nerve root 23. As will be explained below, in one embodiment, the intervertebral disc 12 is accessed through this region by performing a foraminoplasty in which a portion of the inferior vertebra is removed such that surgical instruments or implants can be introduced at this region of the spine. In such a procedure, it is often desired to protect the exiting nerve and the traversing nerve root. Apparatuses and methods for accessing the intervertebral disc through Kambin's triangle may involve performing endoscopic foraminoplasty while protecting the nerve will be discussed in more detail below. Utilizing foraminoplasty to access the intervertebral disc through Kambin's triangle can have several advantages (e.g., less or reduced trauma to the patient) as compared to accessing the intervertebral disc posteriorly or anteriorly as is typically done in the art. In particular, surgical procedures involving posterior access often require removal of the facet joint. For example, transforaminal interbody lumbar fusion (TLIF) typically involves removal of one facet joint to create an expanded access path to the intervertebral disc. Removal of the facet joint can be very painful for the patient, and is associated with increased recovery time. In contrast, accessing the intervertebral disc through Kambin's triangle may advantageously avoid the need to remove the facet joint. As described in more detail below, endoscopic foraminoplasty may provide for expanded access to the intervertebral disc without removal of a facet joint. Sparing the facet joint may reduce patient pain and blood loss associated with the surgical procedure. In addition, sparing the facet joint can advantageously permit the use of certain posterior fixation devices which utilize the facet joint for support (e.g., trans-facet screws, trans-pedicle screws, and/or pedicle screws). In this manner, such posterior fixation devices can be used in combination with interbody devices inserted through the Kambin's triangle.

### Dilation Introducer

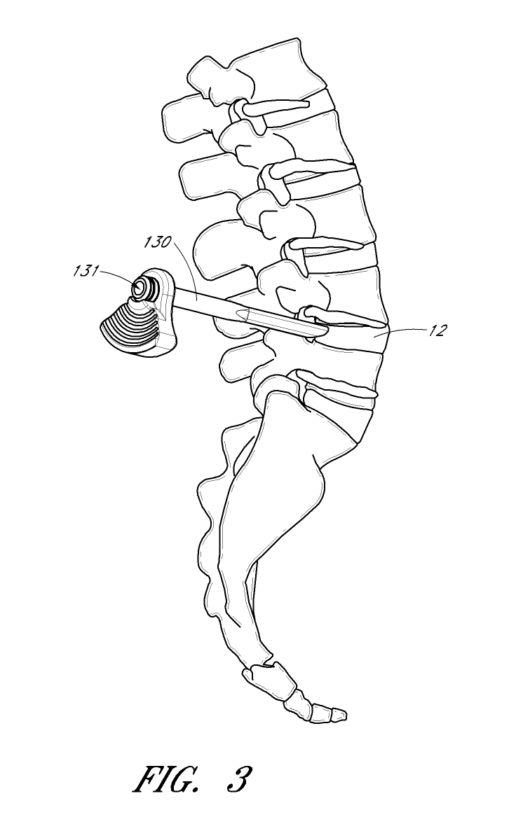

FIGS. 3-8B illustrate an embodiment of a dilation introducer 100 that can be used to perform percutaneous orthopedic surgery. As will be described in detail below, the dilation introducer in the illustrated embodiments can comprise an access cannula and first, second and third dilator tubes. While the illustrated embodiment includes second and third dilator tubes, modified embodiments can include more or less dilator tubes and/or dilator tubes with modified features. It is also anticipated that in some embodiments, the access cannula 130 can be eliminated from the introducer or modified.

FIG. 3 illustrates an embodiment of the access cannula 130, which is shown in a position for performing surgery on an intervertebral disc, for instance transforaminal lumbar interbody fusion. The access cannula 130 in the illustrated embodiment has an inner lumen 131 that allows for surgical instruments and devices to pass through it to access the intervertebral disc 12. The distal tip of the cannula can be oriented such that surgical instruments have access to the intervertebral disc without contacting with the exiting nerve. The position shown in FIG. 3 can be achieved by following the method disclosed herein, discussed in more detail below.

In various embodiments described herein, a first dilator tube may be inserted into the intervertebral space, over which subsequent and larger dilator tubes may be passed. In some embodiments, the first dilator tube may be cannulated to be receive therein a guide wire or K-wire. In some embodiments, the first dilator tube may comprise an access needle, for example between 11 and 18 gauge. In some embodiments, the first dilator tube may comprise a Jamshidi Jamshidi® needle with a removable handle, or a similar device, may be used to initially define a path to the intervertebral disc. With the handle of the Jamshidi® needle removed, a second dilator tube may be advanced over the Jamshidi® needle. In some embodiments, a K-wire or similar device can be inserted through the Jamshidi® needle and/or dilator tubes.

In some embodiments, a first dilator tube may be replaced with a neuro-monitoring needle. The neuro-monitoring needle can include a wire which may be enclosed by a needle cannula, with the wire exposed at the distal tip. The needle cannula may be surrounded by dielectric coating along its length for insulation. For example, the wire can comprise stainless steel and the dielectric coating can comprise parylene. In various embodiments, the coating can be nylon, medthin, or an anodized coating. In some embodiments, a knob may be located on the proximal portion of the neuro-monitoring needle.

The neuro-monitoring needle can be made from several components. The wire portion can be stainless steel coated with dielectric coating of parylene. In various embodiments, the coating can be nylon, medthin, or an anodized coating. The distal tip of the wire can be exposed so that it can transmit current. The needle cannula which covers the wire can also comprise stainless steel coated with parylene or other insulating coating. In some embodiments, this needle cannula could also be described as an exchange tube where once the wire is removed a K-wire could be placed down it and into the disc space. The wire can be attached to a handle at the proximal end ultimately protrude from the handle, serving as the electrode to attach a neuro-monitoring system. In some embodiments, the proximal diameter can be parylene coated, while the rest of the wire can be uncoated to transmit the current.

The wire may comprise a conductive material, such as silver, copper, gold, aluminum, platinum, stainless steel, etc. A constant current may be applied to the wire. The needle cannula may be insulated by dielectric coating. In some embodiments, the coating is need not be dielectric, but rather any sufficiently insulative coating may be used. Alternatively, an insulative sleeve may encase the wire. This arrangement protects the conductive wire at all points except the most distal tip. As the exposed tip of the wire is advanced through the tissue, it continues to be supplied with current. When the tip approaches a nerve, the nerve may be stimulated. The degree of stimulation to the nerve is related to the distance between the distal tip and the nerve. Stimulation of the nerve may be measured by, e.g., visually observing the patient's leg for movement, or by measuring muscle activity through electromyography (EMG) or various other known techniques.

Utilizing this configuration may provide the operator with added guidance as to the positioning of the access needle to the surgical access point and through Kambin's triangle. With each movement, the operator may be alerted when the tip of the needle approaches or comes into contact with a nerve. The operator may use this technique alone or in conjunction with other positioning assistance techniques such as fluoroscopy and tactile feedback. The amount of current applied to the wire may be varied depending on the preferred sensitivity. Naturally, the greater the current supplied, the greater nerve stimulation will result at a given distance from the nerve. In various embodiments the current applied to the conductive wire may not be constant, but rather periodic or irregular. Alternatively, pulses of current may be provided only on demand from the operator.

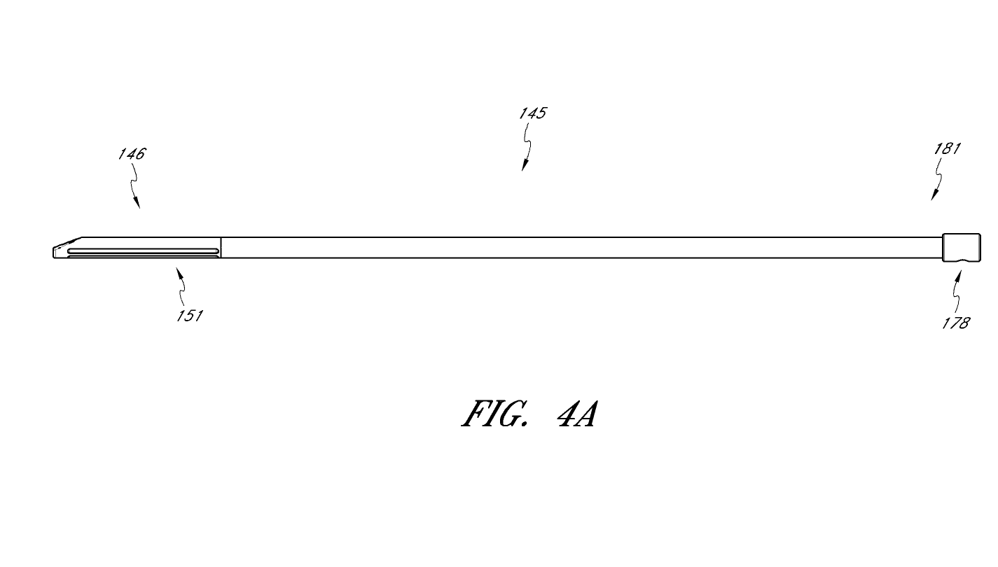

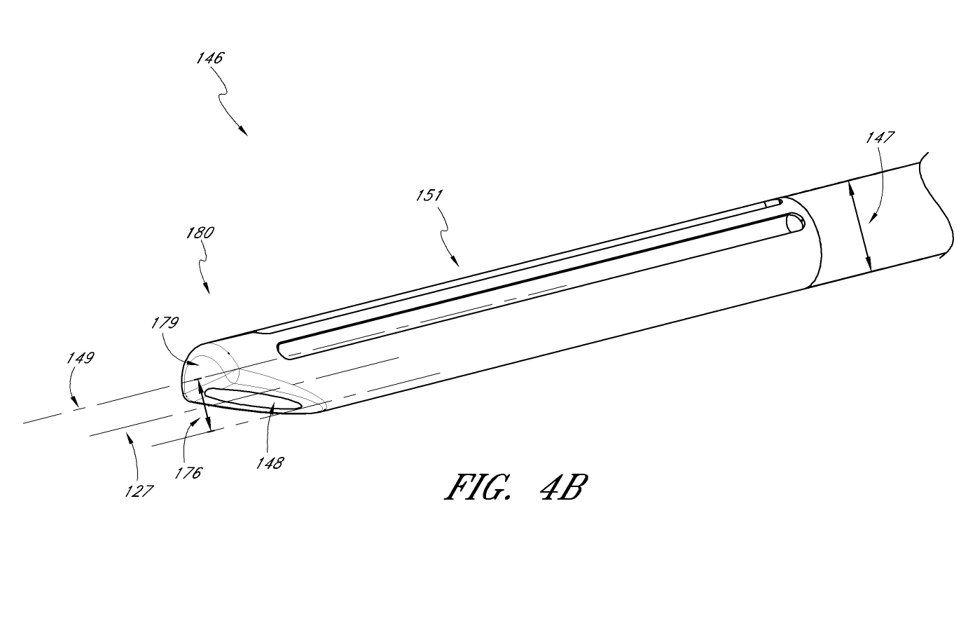

FIGS. 4A-8B illustrate an embodiment of a dilation introducer that can be used to perform percutaneous orthopedic surgery. The dilation introducer 1100 in the illustrated embodiments can comprise an access cannula, and a first, second and third dilator. In some embodiments, the dilation introduce can include more or less dilator tubes and/or dilator tubes with modified features. It is also anticipated that in some embodiments, the access cannula can be eliminated from the introducer or modified.

FIGS. 4A to 4C illustrate an embodiment of the second dilator tube 145. In the embodiment shown the second dilator tube has a distal portion 146, and an outer radius 147. The outer radius may be centered around a second longitudinal axis 149. The second dilator tube includes a second longitudinal lumen 48 with an inner radius 176. The outer radius 142 of the first dilator tube may be nearly equivalent to the inner radius 176 of the second dilator tube, such that the first dilator tube 140 can be slidably received within the second longitudinal lumen 148. The proximal portion 177 of the second dilator tube includes a collar 178.

FIG. 4B shows an enlarged detail view of the distal portion of the second dilator tube 145. The distal portion 146 of the second dilator tube may include a flattened edge 179. This flattened edge 179 advantageously prevents the second dilator tube 145 from penetrating the intervertebral disc 112. The tip 180 of distal portion 146 can have a generally semi-annular cross-section, configured such that when the first dilator tube 140 is received within the second dilator tube 145, the outer radial surface of the first dilator tube 140 is partially exposed at the distal tip 180 of the second dilator tube 145. The opening of the generally semi-annular cross-section of the second dilator tube can be oriented opposite the second longitudinal axis 149 with respect to the longitudinal axis 127 of the second longitudinal lumen.

The distal portion 146 of the second dilator tube may include a conductive pin 188. This conductive pin 188 can be in electrical communication with a proximal electrode, which in turn can be connected to a neuro-monitoring system. As described above with respect to the neuro-monitoring needle, this configuration may provide the operator with added guidance as to the positioning of the second dilator tube to the surgical access point and through Kambin's triangle. With each movement, the operator may be alerted when distal portion 146 of the second dilator tube 145 approaches or comes into contact with a nerve. The operator may use this technique alone or in conjunction with other positioning assistance techniques such as fluoroscopy and tactile feedback. The amount of current applied to the wire may be varied depending on the preferred sensitivity. Naturally, the greater the current supplied, the greater nerve stimulation will result at a given distance from the nerve. In various embodiments the current applied to the conductive wire may not be constant, but rather periodic or irregular. Alternatively, pulses of current may be provided only on demand from the operator.

In some embodiments, the entire second dilator tube 145 except for the exposed conductive pin 188 and a proximal electrode can be coated with dielectric material, for example parylene or nylon, anodization-type coating, or medthin. Accordingly, in such embodiments current can be applied to the proximal electrode, and due to the dielectric coating, no stimulation can exit the second dilator tube until reaching the exposed conductive pin 188 at the distal end.

When a first dilator tube is received within the second dilator tube 145, the longitudinal axis 127 of the second longitudinal lumen is essentially aligned with a first longitudinal axis of the first dilator tube. Additionally, the second dilator tube 145 can include cutting flutes or ridges 151 on one side, located opposite the opening of the generally semi-annular cross-section of the second dilator tube 145. In other embodiments, the cutting flutes 151 may be replaced with a coarse surface (e.g., knurling, sharp edges, abrasive members, etc.) which, when rotated or slid (e.g., back and forth) against bone, will create a recess therein. As noted above, other mechanisms for removing bone can be used, and the cutting flutes are shown here by way of example only. As can be seen in FIG. 4B, the inner lumen 148 of the second dilator tube 145 can be off-center. In this configuration, the cutting flutes 151 are further from the axis of rotation than the side opposite the cutting flutes. This is particularly advantageous for performing foraminoplasty while protecting the exiting nerve, as will be discussed in more detail below.

FIG. 4C shows an enlarged detail view of the proximal portion 177 of the second dilator tube 145. The collar 178 includes an aperture 181 which may be used in conjunction with the third dilator tube, as described in detail below. In alternative embodiments, the aperture 181 may be instead replaced with a circumferentially oriented groove.

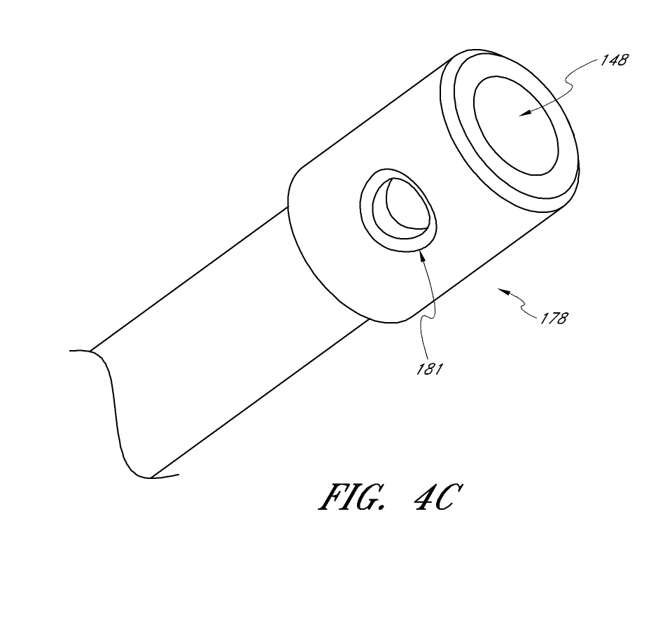

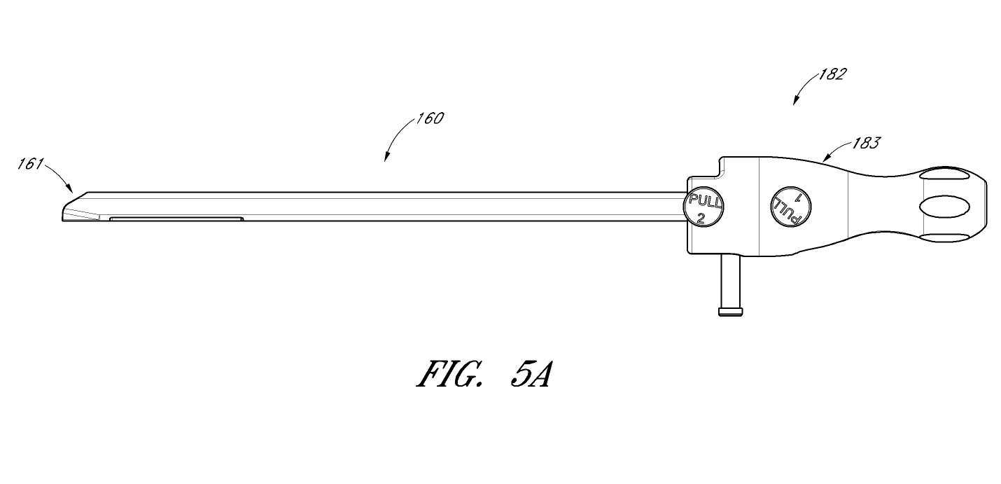

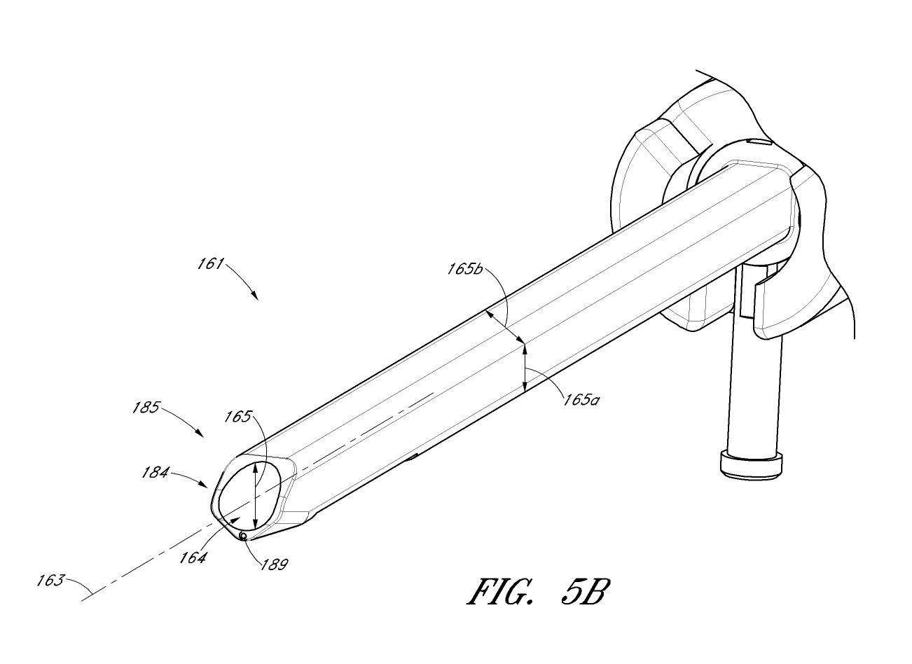

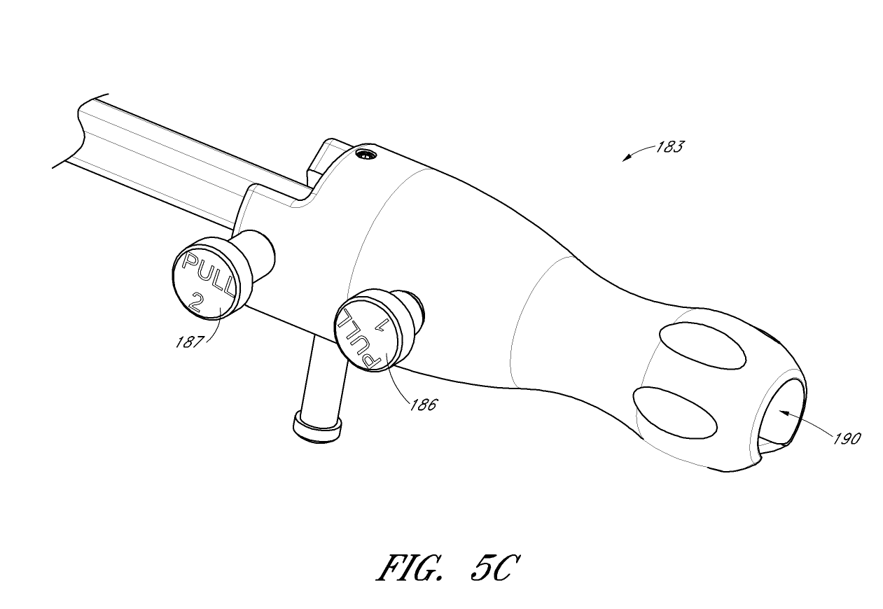

FIGS. 5A to 5D illustrate an embodiment of the third dilator tube 160, which can be configured to be slidably introduced over the second dilator tube 145. The third dilator tube 160 can include a distal portion 161 and an outer surface 162 that is substantially rectangular (i.e., rectangular) in cross-section. The substantially rectangular cross-section of outer surface 162 is centered around a third longitudinal axis 163. The third dilator tube 160 also includes a third longitudinal lumen 164 having a third inner radius 165 centered around the third longitudinal axis 163. The third lumen 164 can be configured to removably receive the second dilator tube 145 for slidable movement within the third lumen 164. For example, as illustrated, the third lumen 164 can be substantially circular in cross-section. When the second dilator tube 145 is removably received within the third lumen 164, the second longitudinal axis 149 essentially aligns with the longitudinal axis 169 of the inner lumen 164 of the third dilator tube 160. The proximal portion 182 includes a handle assembly 183.

The terms “approximately”, “about”, and “substantially” as used herein represent an amount or characteristic close to the stated amount or characteristic that still performs a desired function or achieves a desired result. For example, the terms “approximately”, “about”, and “substantially” may refer to an amount that is within less than 10% of, within less than 5% of, within less than 1% of, within less than 0.1% of, and within less than 0.01% of the stated amount characteristic. The term “up to about” as used herein has its ordinary meaning as known to those skilled in the art and may include 0 wt. %, minimum or trace wt. %, the given wt. %, and all wt. % in between.

Accordingly, a substantially rectangular cross-section can in certain embodiments include arrangements in which the adjacent sides of the rectangular cross-section within 10%, 5%, 1%. 0.1% or 0.01% of 90 degrees of each other. A rectangular cross-section can in certain embodiments include rounded or otherwise modified edges. In addition, in certain embodiments a substantially rectangular cross-section can include four substantially flat sides. However, such substantially flat sides can include ridges, textures, etc. that deviate from the generally flat nature of a side.

In addition, while certain embodiment is described as being “substantially rectangular” in other embodiments such the access cannula has at least one flat side. In another embodiment, the access cannula has at least two flat sides that can be positioned adjacent to each other or opposing each other. In another embodiment, the access cannula has at least two flat sides that are substantially at right angles to each other. In another embodiment, the access cannula has at least three flat sides in which adjacent sides are at substantially at right angles to each other. The term “substantially flat” can include arrangements in which deviations along surface are within 10%, 5%, 1%. 0.1% or 0.01% of the length or width of the surface.

FIG. 5B shows an enlarged detail view of the distal portion of the third dilator tube of FIG. 5A. The distal portion 161 of the third dilator tube may include a flattened edge 185. This flattened edge 185 advantageously prevents the third dilator tube 160 from penetrating the intervertebral disc 112. The tip 184 of the distal portion 161 has a generally semi-annular cross-section. In some embodiments, cutting flutes for reaming bone can be located opposite the opening of the semi-annular cross-section. As with the second dilator tube, in other embodiments cutting flutes may be replaced or used in combination with a coarse or other cutting or abrading surface which, when rotated or slid against bone, will create a recess therein. As can be seen in FIG. 5B, the longitudinal lumen 164 of the third dilator tube 160 may be centered around longitudinal axis 163. In other embodiments, the lumen may be off-center.

With continuing reference to FIG. 5B, the outer surface of the third dilator tube is substantially rectangular in cross-section, having a height 165a and a width 165b. In some embodiments, the cross-section may be substantially square, in which case the height 165a and width 165b are approximately equal. The outer surface of the third dilator tube can be centered around the third longitudinal axis 163. As noted above, the inner longitudinal lumen 164 may also be centered around the third longitudinal axis 163.

The distal portion 161 of the third dilator tube may include a conductive pin 189. This conductive pin 189 can be in electrical communication with a proximal electrode, which in turn can be connected to a neuro-monitoring system. As described above with respect to the second dilator tube, this configuration may provide the operator with added guidance as to the positioning of the third dilator tube to the surgical access point and through Kambin's triangle. With each movement, the operator may be alerted when distal portion 161 of the third dilator tube 160 approaches or comes into contact with a nerve. In some embodiments, the entire third dilator tube 160 except for the exposed conductive pin 189 and a proximal electrode can be coated with dielectric or insulating material, for example parylene or nylon, an anodization-type coating, or medthin. Accordingly, in such embodiments current can be applied to the proximal electrode, and due to the dielectric coating, no stimulation can exit the third dilator tube until reaching the exposed conductive pin 189 at the distal end.

FIG. 5C shows an enlarged detail view of the proximal portion 182 of the third dilator tube 160. The proximal portion 182 includes a handle assembly 183. A first latching button 186 may be configured for constraining the movement of the third dilator tube relative to the second dilator tube, as described in more detail below. In various embodiments, the latching button 186 may constrain slidable movement, rotational movement, or both. A second latching button 187 may be located distal the first latching button 186, and may be configured to constrain the movement of the access cannula relative to the third dilator tube, as described in more detail below.

FIG. 5D shows a front view of the third dilator tube 160. As illustrated, the longitudinal lumen 164 has a substantially circular cross-section, while the outer surface 167 of the third dilator tube 160 is substantially rectangular.

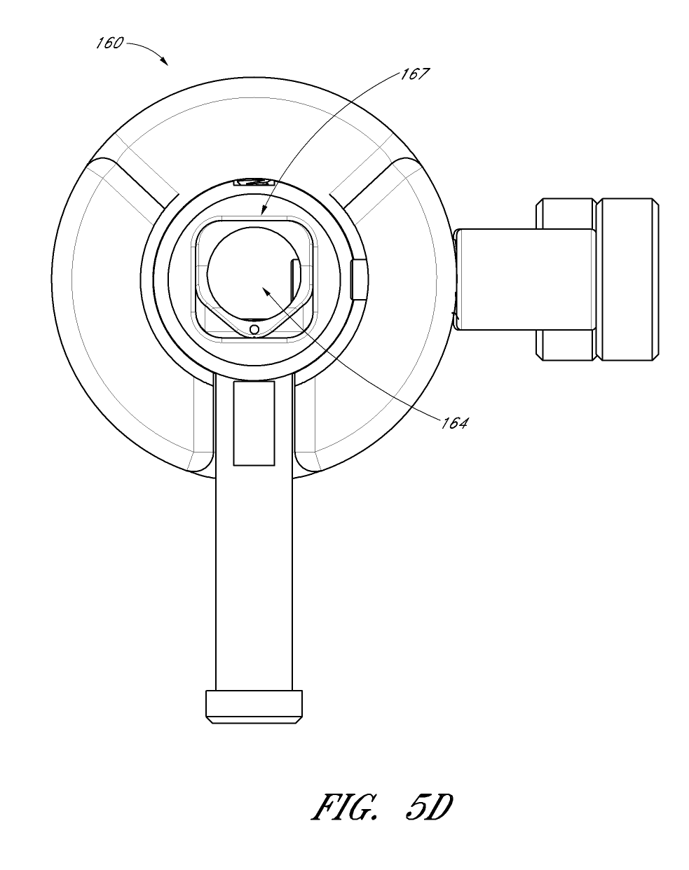

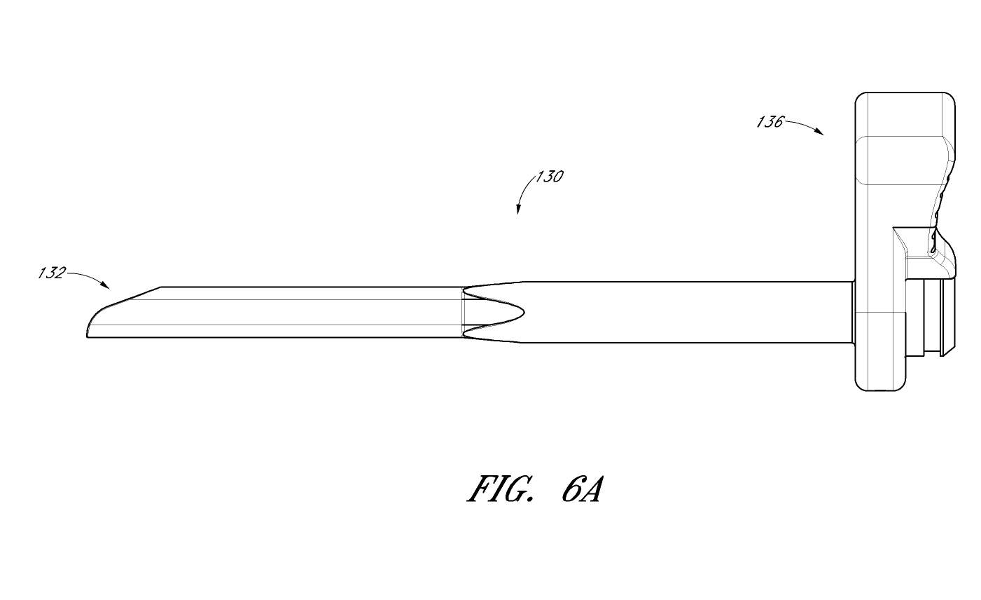

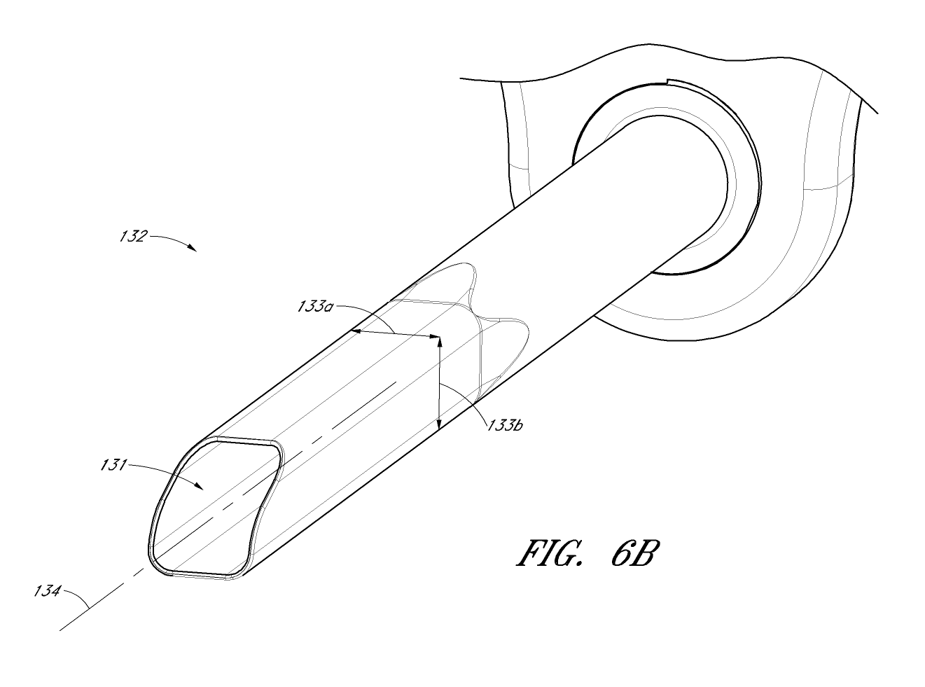

FIGS. 6A to 6C illustrate an embodiment of the access cannula 130, which can be configured to be advanced over the third dilator tube 160. The access cannula 130 has a distal portion 132, a fourth longitudinal axis 134, and a fourth longitudinal lumen 131. As with the outer surface of the third dilator tube 160, the lumen 131 of the access cannula 130 can have a substantially rectangular cross-section, and can have a width 133a and a height 133b. The access cannula 130 may be configured to removably receive the third dilator tube (not shown) for slidable movement within the third lumen. A handle 136 allows for rotation of the access cannula 130. In the illustrated embodiment, the outer surface of the third dilator tube 160 and the inner lumen 131 of the access cannula 130 are both substantially rectangular in cross-section. As such, the third dilator tube 160, in this configuration, cannot be rotated with respect to the access cannula 130. The access cannula 130 can slide proximally and distally relative to the third dilator tube 130, but their relative rotational orientation may remain fixed. Even while fixed with respect to one another, however, both the access cannula 130 and the third dilator tube 130 may, together, rotate with respect to the second dilator tube 145 and/or the first dilator tube 140.

FIG. 6B shows an enlarged detail view of the distal portion of the access cannula of FIG. 6A. The distal portion 132 can have a beveled or tapered shape, in which the cross-section is a partial rectangle or U-shape. In the embodiment shown, the fourth longitudinal lumen may be centered with respect to the outer surface of the access cannula, in contrast to the second and third dilator tubes. In other embodiments, however, the access cannula may also have a longitudinal lumen that is off-center with respect to the outer surface. In yet another embodiment, the access cannula need not be limited to a substantially rectangular outer surface. The outer surface could, for instance, have an elliptical, polygonal, or other cross-sectional shape. In some embodiments, a portion of the outer surface of the access cannula may include retention features. Such retention features can help the access cannula retain its position once inserted into the intervertebral space or is positioned near the intervertebral space. In various embodiments, the retention features can be grooves, teeth, protrusions, or other abrasive features. In some embodiments, the retention features can be disposed in the distal portion of the access cannula. In some embodiments, retention features can be limited to top and bottom outer surfaces of the access cannula. Various other configurations are possible.

In some embodiments, the access cannula may be coated with a dielectric or insulating coating, other than a first uncoated area in the distal region and a second uncoated area in the proximal region. The distal uncoated area may be, for example, a small circle or in other embodiments may be an uncoated line. In some embodiments, an uncoated line can be approximately 1 mm wide and approximately 15-30 mm in length. Once the access cannula is in its final position, the surgeon can stimulate via the uncoated proximal region to get an idea of how far away the outer walls of the cannula are in relation to the exiting nerve. As described previously, the dielectric or insulating coating can be, for example, parylene, nylon, an anodization-type coating, medthin, or other appropriate coating.

FIG. 6C shows an enlarged detail view of the proximal portion 193 of the access cannula of FIG. 6A. The proximal grip 136 may provide additional leverage while advancing the access cannula over the third dilator tube. The proximal grip 136 includes a larger diameter portion 198 and a smaller diameter portion 199. The smaller diameter portion 199 includes a circumferential channel 1107 for use in interlocking with the third dilator tube, as discussed in detail below. A locking pinhole 1104 can receive the locking pin 1103 on the third dilator tube, thereby restraining rotational movement of the access cannula 130 relative to the third dilator tube 160. As noted above, in some embodiments the access cannula 130 and the third dilator tube 160 cannot be rotated relative to one another due to the shape and dimensions of the outer surface of the third dilator tube 160.

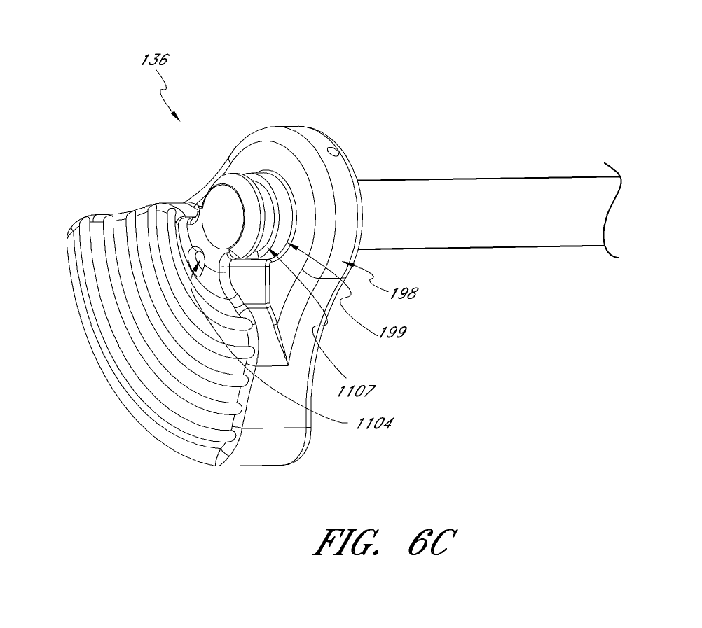

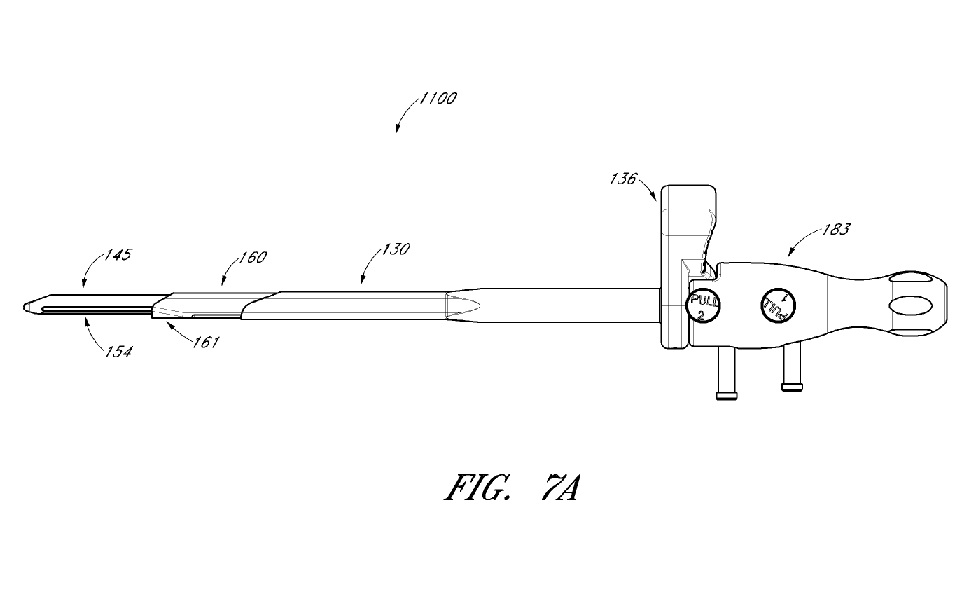

FIGS. 7A to 7C illustrate one embodiment of the dilation introducer 1100 in an assembled configuration. As shown, the access cannula 130 can be positioned over the third dilator tube 160, which can be positioned over the second dilator tube 145, which in turn can be positioned over the first dilator tube 140. The handle assembly 183 of the third dilator tube may be in a locked configuration with the proximal grip 136 of the access cannula can be locked together to constrain slidable. Additionally, the second dilator tube 145 may be locked together with the third dilator tube to constrain slidable movement, while still allowing the second dilator tube 145 to rotate with respect to the third dilator tube. Alternatively, the second dilator tube may be in a locked configuration preventing both slidable and rotational movement with respect to the third dilator tube 160. The third dilator tube 160 can be advanced distally until the distal portion 161 of the third dilator tube aligns with the distal portion 146 of the second dilator tube. Further, the access cannula 130 may also be advanced so that the distal portion 132 aligns with the distal portions 146, 161 of the second and third dilator tubes. The second dilator tube 145 may have cutting flutes 151 on distal portion 146. As can be seen, the second and third longitudinal axes 149 and 163 here are coincident, and are parallel to and laterally offset from first longitudinal axis 144.

In certain embodiments, the first, second and third dilator tubes 140, 145, 160 along with the access cannula 130 can be provided with additional stops that engage the proximal grip 136 of the access cannula and the handle assembly 183 of the third dilator tube described above. For example, in one embodiment, notches or detents can be provided that engage the proximal grip 136 or handle assembly 183 when one tube is advanced distally and reaches a specific location (e.g., end point). In this manner, forward movement of a tube or cannula can be limited once the tube or cannula is advanced to a desired location

FIG. 7B shows an enlarged detail view of the distal portion of the dilation introducer of FIG. 7A. The distal portions 146, 161 of each of the second and third dilator tubes 145, 160, may have generally semi-annular cross-sections, while the distal portion 132 of the access cannula 130 may have a generally semi-rectangular or U-shaped cross-section. The distal portions 146, 161 of the second and third dilator tubes 145, 160 in the illustrated embodiment can have flattened edges 179, 185 to prevent penetration into the intervertebral disc as each dilator tube is advanced.

As noted above, each of the second and third dilator tubes, and the access cannula can have exposed conductive portions configured to be in electrical communication with a neuro-monitoring system. As the dilator tube or access cannula is advanced through the tissue and towards the access site, nerve stimulation may be monitored as described above. The current supplied to each of the second and third dilator tubes and to the access cannula may be controlled independently, so that when nerve stimulation is observed, the operator may supply current separately to each wire to determine which wire or wires are nearest to the nerve. Alternatively, current may be supplied only to one wire at any given point in the procedure. For example, the current may be supplied to the wire associated with the dilator tube or access cannula that is being moved at that point in the operation.

In some embodiments, the second and third dilator tubes can comprise aluminum that has been anodized and then coated with parylene. Certain areas of the second and third dilator tubes can be masked from the anodization and parylene coating so that they can transmit the current. For example, the distal tips of the second and third dilator tubes can be exposed so as to conduct current therethrough. The exposed portions can be passivated to resist rusting, pitting, or corrosion. The exposed portions can be made by using a stainless steel pin pressed into the second and third dilator tubes. The pin can aid in locating the second and third dilator tubes on x-ray or fluoroscopy, and additionally can facilitate the transmission of current through the second and third dilator tubes to the area of contact. Electrode attachments for the second and third dilator tubes can be coated with parylene on the proximal larger diameter to prevent current from flowing into the user. The rest of the electrode can be uncoated, but passivated to resist rusting, pitting, or corrosion. The electrodes can attach such that the current is transmitted to the internal area of the second and third dilator tubes so that it can be transmitted distally through the exposed areas on the tips of the tubes. These tubes may be attached to Radel handles, which being a polymer are also insulators. The third dilator tube can be made from stainless steel, coated with nylon or other polymer, such as Teflon, followed by a parylene coating. In embodiments in which the dilator tube comprises stainless steel, no additional x-ray marker is required.

FIG. 7C shows an enlarged detail view of the proximal portion of the dilation introducer of FIG. 7A. The proximal grip 136 of the access cannula 130 is shown in a locked configuration with the handle assembly 183 of the third dilator tube 160. The smaller diameter portion (not shown) may be received within an overhanging lip on the distal end of the handle assembly 183. Latching buttons 186, 187 constrain movement of the third dilator tube relative to the second dilator tube, and of the access cannula relative to the third dilator tube, respectively. In some embodiments, the first dilator tube may be fastened to the handle assembly 183 by means of a threaded engagement between the proximal head of the first dilator tube and the handle assembly 183. In such configurations, this fastening may constrain both rotational and slidable movement of the first dilator tube relative to the third dilator tube. In various embodiments, the first dilator tube may be affixed to the handle assembly 183 by other means that allow for free rotational movement, free slidable movement, or both.

As noted above, the third dilator tube 160 and the access cannula 130 each have outer surfaces that are substantially rectangular in cross-section. It is understood that the term “rectangular” as used herein also includes a square shape. This stands in contrast to the substantially rounded outer surfaces of the second dilator tube 145. In some embodiments, the shape and dimensions of the lumen of the access cannula 130 can be configured to receive an intervertebral implant therethrough. In particular, an intervertebral implant having a substantially rectangular cross-section can be passed through the lumen of the access cannula. Due to the substantially rectangular shape, the total cross-sectional size of the lumen can be reduced relative to rounded configurations. For example, in some embodiments the height and width of the lumen can each be reduced by about 2.2 mm relative to a rounded configuration.

In some embodiments, the reduction in these dimensions can allow reduce the need for foraminoplasty and/or can reduce the risk of damaging the traversing nerve root during the procedure. Additionally, the reduced dimensions may aid in accessing particularly tight disc spaces, such as in the L5/S1 region. In some embodiments, the substantially rectangular shape of the third dilator tube 160 can aid the foraminoplasty procedure. The sharper edges, as compared to the rounded configuration, may more readily remove bone to expand the foramen. In some embodiments, the substantially rectangular cross-section of the access cannula lumen advantageously facilitates docking the access cannula within the disc space. The position of the access cannula may thereby be more easily retained, allowing for accurate and precise insertion of intervertebral implants into the disc space.

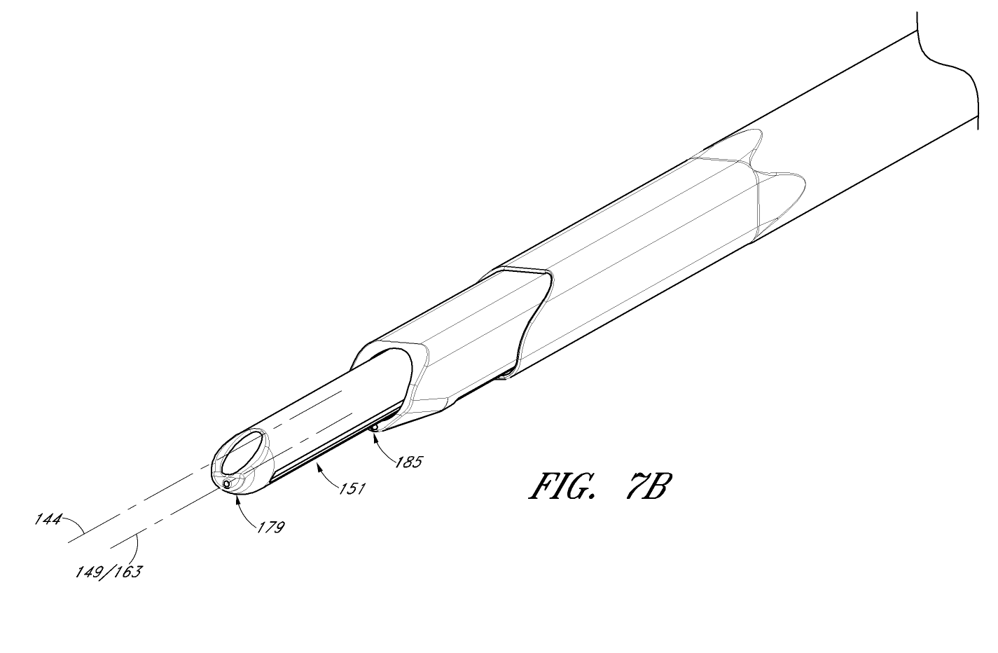

Referring to FIGS. 8A and 8B, a dilation introducer 1100 is shown in a locked assembled configuration. The dilation introducer 1100 includes a second dilator tube 145, a third dilator tube 160, and an access cannula 130. The second dilator tube 145 has a distal tip 180 with a flattened edge 179, a proximal portion 177 with a collar 178, and a longitudinal lumen 148. As described above, first dilator tube, Jamshidi, access needle or similar device may be removably received within the second dilator tube 145.

The third dilator tube 160 has a distal tip 184 with a flattened edge 185, a proximal portion 182 with a handle assembly 183, and a longitudinal lumen 164. The second dilator tube 145 may be removably received in the longitudinal lumen 164 of the third dilator tube 160 for slidable movement within the third dilator tube 160. The second and third dilator tubes may be connected together in a locked configuration with a first latching button 186 disposed on the handle assembly 183 of the third dilator tube 160 and extending through a first aperture 1105 in the handle assembly 183 of the third dilator tube 160, so that the first latching button 186 may be moveable between a radially inward locking position (arrow 1101) and a radially outward unlocking position (arrow 1102).

The distal end 196 of the first latching button may be removably received in aperture 181 of the second dilator tube 145 so as to engage and lock the second and third dilators together in the locking position. Alternatively, the latching button may be received in a circumferentially oriented groove of the second dilator tube, which may or may not extend completely around the second dilator tube. The first latching button 186 may be pulled radially outwardly to release the second dilator tube 145, to allow the third dilator tube 160 to slide with respect to the second dilator tube 145.

The access cannula 130 has a distal portion 161, a proximal portion 193, a proximal grip 136, and longitudinal lumen 164. The third dilator tube 160 may be removably received within the access cannula 130 for slidable movement within the longitudinal lumen 131 of the access cannula 130. The third dilator tube 160 and the access cannula 130 also have a locked configuration in which the access cannula 130 may be not permitted to slidably telescope over the third dilator tube 160.

The proximal portion 193 of the access cannula 130 includes a proximal grip 136 with a larger diameter portion 198 and a smaller diameter portion 199. The smaller diameter portion 199 may be sized to fit under an overhanging lip 191 of the third dilator tube, when the longitudinal axes of the third dilator tube and access cannula may be aligned. There may be a circumferentially oriented channel 1107 in the exterior of the smaller diameter portion 919 for receiving a distal end 197 of a second latching button 187. The circumferentially oriented channel 1107 does not need to extend completely around the exterior of the smaller diameter portion 199.

The third dilator tube 160 and the access cannula 130 may be connected together in a locked configuration with the second latching button 187 disposed on the overhanging lip 191 of the handle assembly 183 of the third dilator tube 160. The second latching button extends through an aperture 1106 in the overhanging lip 191 of the handle assembly 183 and may be movable between a radially inward locking position (arrow 194) and a radially outward unlocking position (arrow 195). The distal end 197 of the second latching button 187 may be removably received in the channel 107 located in the smaller diameter portion 199 of the access cannula 130, in the locking position, to lock the third dilator tube 160 and the access cannula 130 in the locked assembled configuration. The second latching button 187 may be pulled radially outward to release the access cannula 130 to slide to the unlocked configuration. Furthermore, the second and third dilator tubes 140, 145 may be removed together as a unit from the access cannula 130. In other words, the second dilator tube 145 can be removed from the access cannula 130 by unlocking the second latching button 187 alone. An advantage of this embodiment is that the latching buttons 186, 187 may be both removable from the surgical field with the release of the third dilator tube from the access cannula 130.

The access cannula being free of protuberances, such as the latching buttons, is less likely to catch surgical sponges and sutures, for example, on the dilation introducer.

### Method of Use

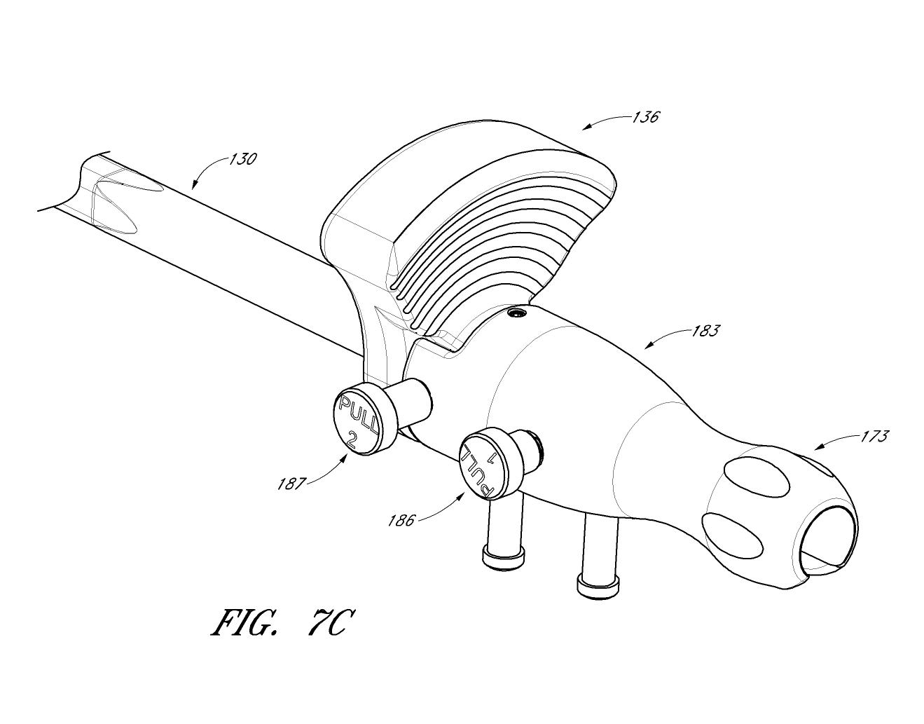

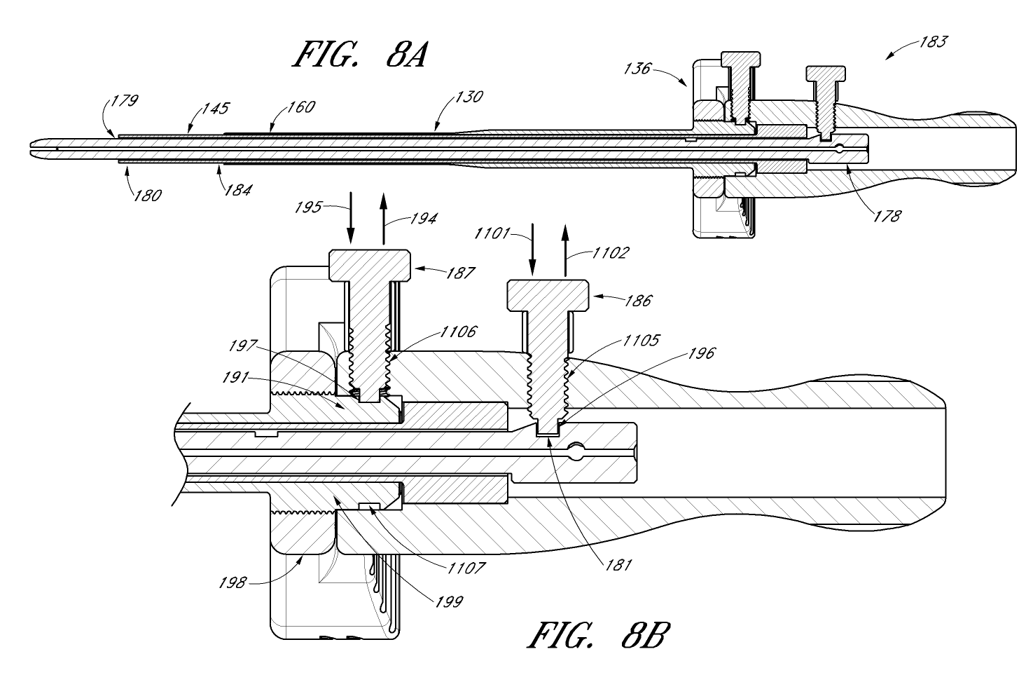

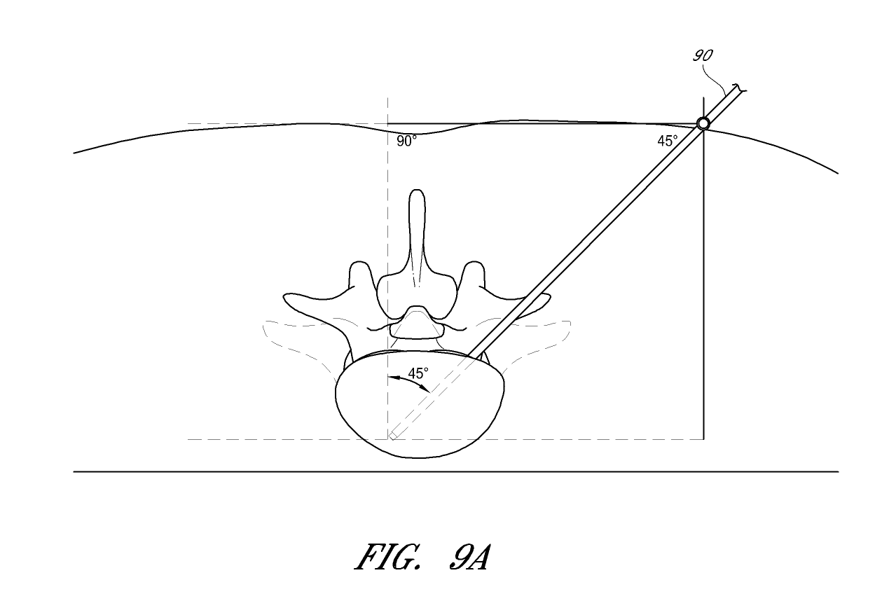

FIGS. 9A-13C illustrate one embodiment of a method of performing percutaneous orthopedic surgery using the dilation introducer. In some embodiments, a trocar or access needle can be inserted into the intervertebral space. In some embodiments, the insertion point and access trajectory can first be determined. For example, a patient may lie face down on a surgical frame to facilitate a lordotic position of the lumbar spine. With aid of a lateral x-ray or other imaging system, a K-wire (or equivalent) can be laid beside the patient and placed to the depth of optimal insertion for the intervertebral implant. Intersection with the skin can be marked on the K-wire (or equivalent). With the aid of an anteroposterior x-ray or other imaging system, the K-wire (or equivalent) can be laid on top of the patient, aligned with the disc in a view that allows for the end plates to be parallel (e.g., Ferguson View or Reverse Ferguson, as applicable). The distance between the midline and the previously marked point on the K-wire can define the insertion point.

As illustrated in FIGS. 9A-9C, a small skin incision can be made defining a trajectory into the disc can be between 45 and 55 degrees. Next, a trocar 90 can be placed into the center of the disc 12 of the level to be treated, up to but not through the distal annulus. Alternatively, an 11 gauge to 18 gauge access needle, or a first dilator tube can be used. As shown in FIGS. 9B-C, the inner stylet 92 of the trocar (if present) can be removed while maintaining the outer sheath 94 in place within the disc 12. Alternatively, a K-wire can be inserted into the disc and the outer sheath may be removed. Next, a dilation introducer 96 can be placed over the outer sheath 94 of the trocar (or over the K-wire, if applicable). The dilation introducer 96 can be aligned so that the smooth edges are oriented towards the exiting nerve root and the foramen. In some embodiments, the dilation introducer 96 can include at least second and third dilator tubes, each having cutting flutes adapted to perform foraminoplasty for improved access to the disc space. In some embodiments, the second dilator tubes may be rotated within +/−45 degrees around the longitudinal axis so that the cutting flutes do not contact the exiting nerve.

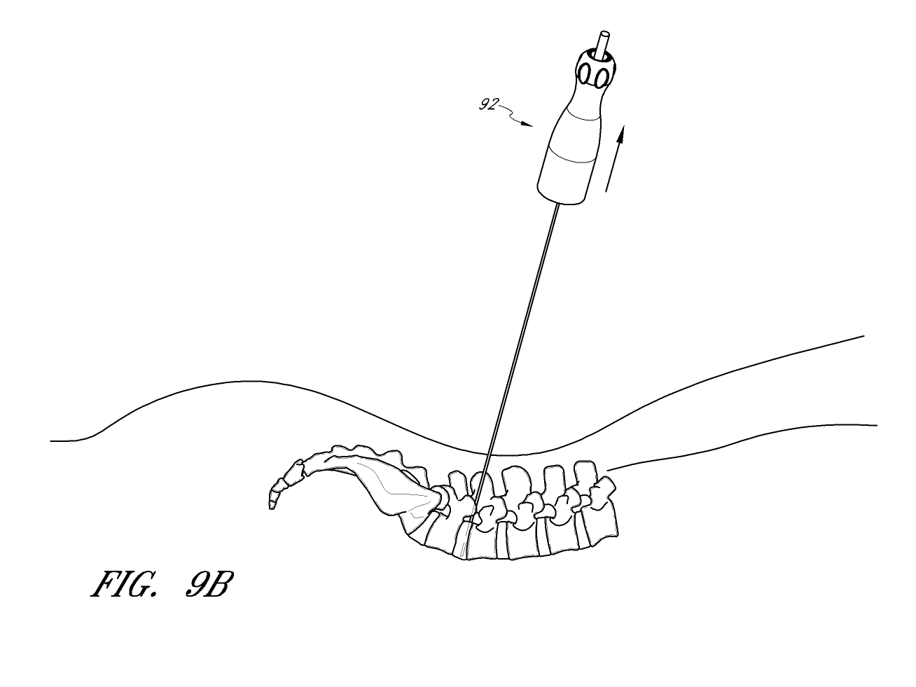

With initial reference to FIG. 10A, the dilation introducer can be advanced until the first dilator tube passes through Kambin's triangle 20, and the distal portion abuts or even penetrates the intervertebral disc 12. In one arrangement, the second dilator tube 145 can then be advanced over the first dilator tube 140 until the distal portion 146 of the second dilator tube abuts but does not enter the intervertebral disc 12.

In another alternative embodiment, the first dilator tube may be omitted. Instead, a Jamshidi® needle with a removable handle or similar device may be used. In such an embodiment, the Jamshidi® needle may be first introduced to abut or enter the intervertebral disc, after which the handle may be removed. Optionally, a K-wire may be inserted into the Jamshidi® needle after it is in position either abutting or partially penetrating the intervertebral disc. The second dilator tube may then be advanced over the Jamshidi® needle.

FIG. 10B shows an enlarged detail of the second dilator tube 145 introduced over the first dilator tube 140. The distal portion 46 of the second dilator tube 145 can have a semi-annular cross-section with an opening that forms a recess with respect to the leading edge of the tube 145. The second dilator tube 145 can be oriented for advancement over the first dilator tube 140 such that the opening of the semi-annular cross-section faces the exiting nerve 21. This technique advantageously limits and/or eliminates contact with the exiting nerve. The distal portion 146 of the second dilator tube opposite the opening of the semi-annular cross-section abuts the inferior vertebrae 22. The cutting flutes (not shown) are positioned against the inferior vertebrae 22. The second dilator tube 145 may be rotated slightly back and forth, such that the cutting flutes create a recess in the inferior vertebrae 22, making room for introduction of the third dilator tube. When rotating the second dilator tube, care is taken to minimize any trauma inflicted upon the exiting nerve. Accordingly, in the illustrated embodiment, the tube 145 can be used to remove bone on a side of the tube 145 generally opposite of the nerve 21.

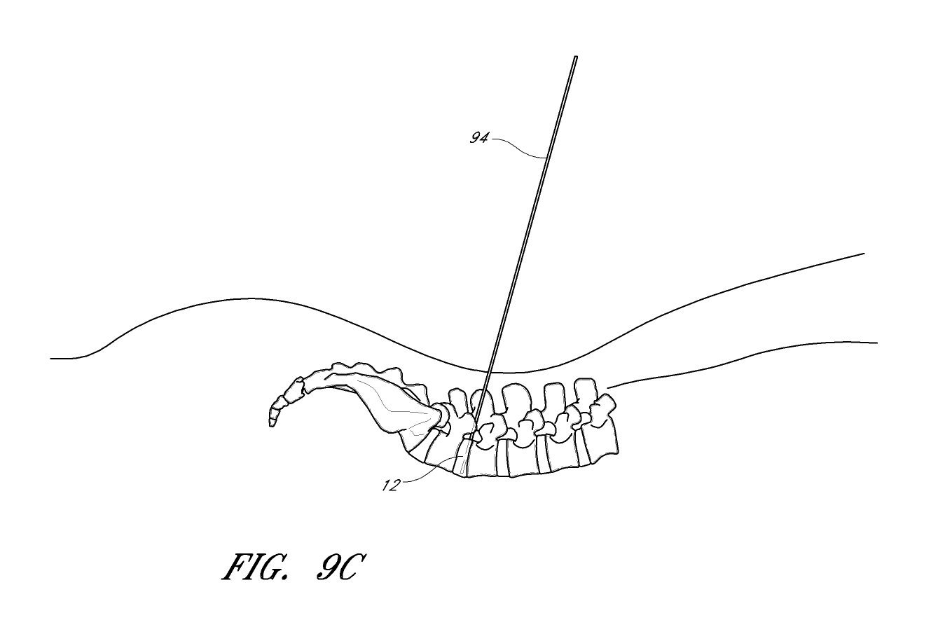

With reference now to FIG. 11, the third dilator tube 160 can be introduced over the second dilator tube 145. In one arrangement, the distal portion of the third dilator tube 160 abuts but does not enter the intervertebral disc. In the illustrated embodiment, a flattened edge of the distal portion can help ensure that the third dilator tube 160 does not penetrate the intervertebral disc or limit such penetration. As with the second dilator tube, the opening of the semi-annular cross-section of the distal portion of the third dilator tube can be positioned to face the exiting nerve (not shown). Contact between the third dilator tube 160 and the nerve can thereby be minimized or eliminated. The cutting flutes 168 of the third dilator tube can be positioned opposite the opening of the semi-annular cross-section, and abut the inferior vertebrae 22. The third dilator tube 160 may be rotated slightly back and forth, such that the cutting flutes create a further recess in the inferior vertebrae 22, making room for introduction of the access cannula. Again, care should be taken during the rotation of the third dilator tube to ensure that the exiting nerve is not injured thereby. Accordingly, the third dilator tube can be can be used to remove bone on a side of the tube 60 generally opposite of the nerve 21.

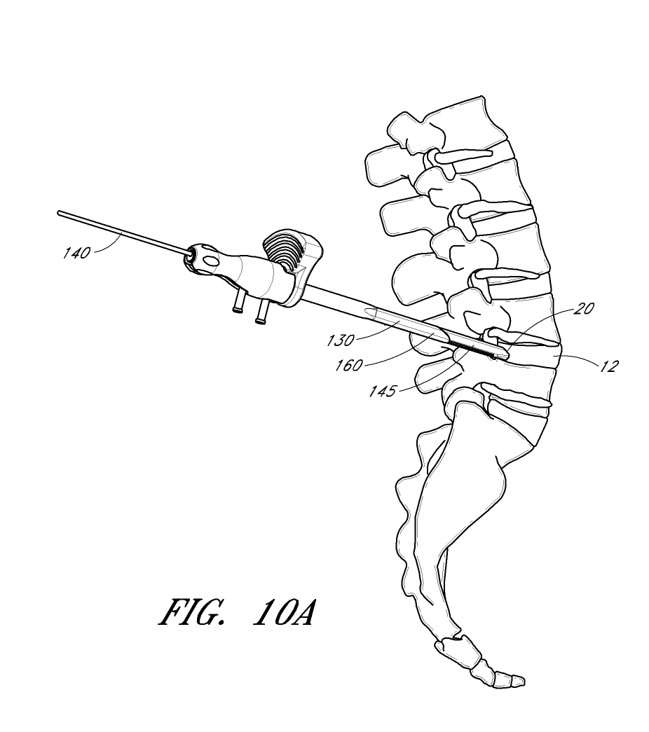

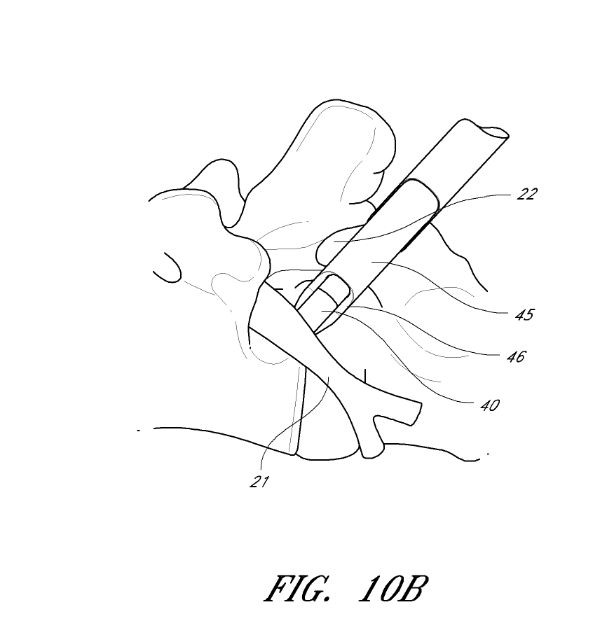

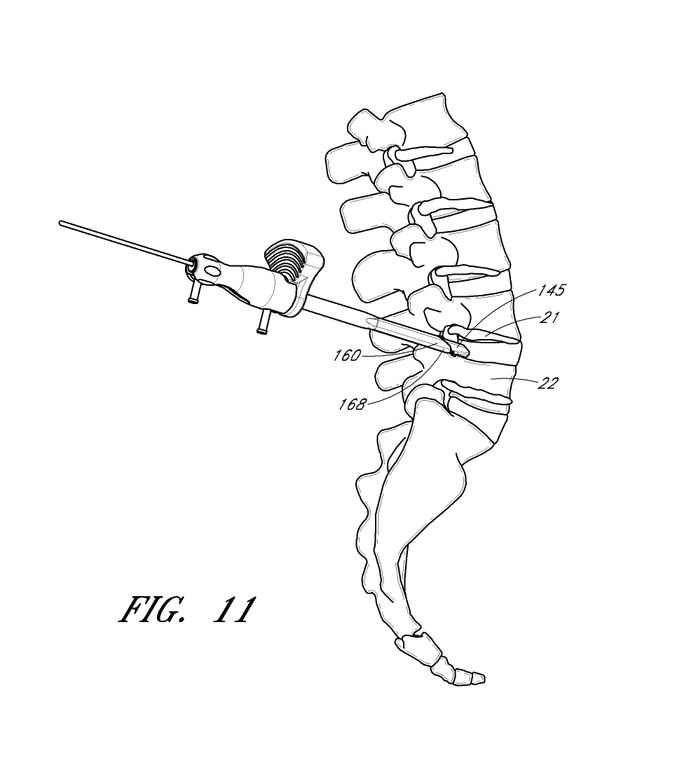

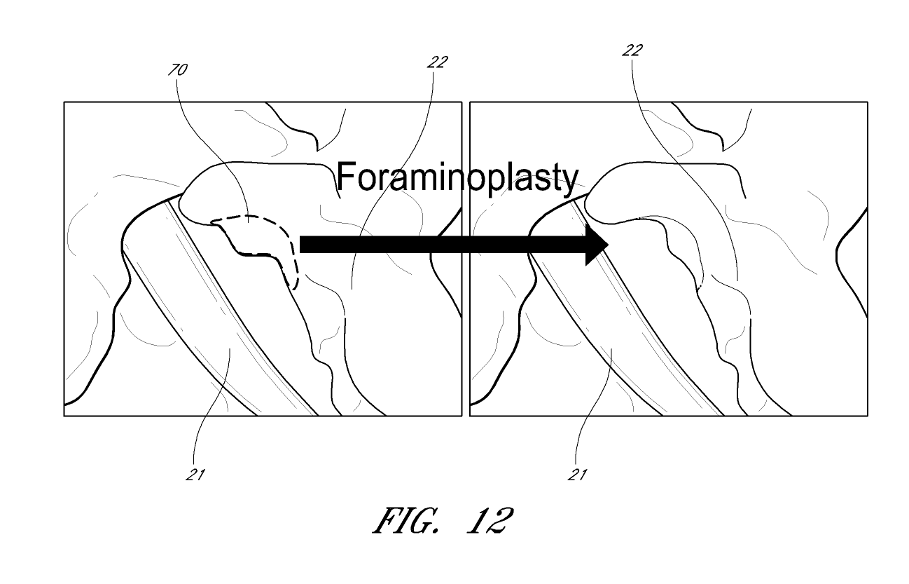

FIG. 12 shows the access area before and after the second and third dilator tubes 145, 160 are rotated to create a recess in the inferior vertebrae 22. The area 70 in the left image demarcated by a dashed line is the portion of bone that can be removed by the second and third dilator tubes 145, 160. This foraminoplasty permits the access cannula to be introduced without disturbing the exiting nerve 21. The method described is not limited by the precise location of the recess shown in FIG. 12. In general, a recess may be formed anywhere along the superior border of the inferior vertebrae 22, in order to provide improved access for a dilation introducer.

FIG. 13A shows the access cannula 130 introduced over the third dilator tube 160. The distal portion of the access cannula 130 abuts but does not enter the intervertebral disc 12. In one embodiment, the distal portion can be equipped with flattened edges to guard against insertion into the intervertebral disc. As with the second and third dilator tubes 145, 160, the opening of the semi-annular cross-section of the distal portion of the access cannula 130 can be positioned initially to face the exiting nerve 21. Contact between the access cannula 130 and the exiting nerve can thereby be minimized during insertion.

As can be seen in FIG. 13B, the access cannula 130 can then be rotated such that the opening of the semi-annular cross-section faces opposite the exiting nerve 21. Since, unlike the second and third dilator tubes 145, 160, the outer surface of the access cannula is smooth, trauma to the exiting nerve may be minimized during this rotation.

Referring now to FIG. 13C, once the access cannula 130 is in position, which in one embodiment comprising until the distal portion abuts the intervertebral disc 12, the cannula 130 can be rotated so that the opening of the semi-annular cross-section faces opposite the exiting nerve 21, the first, second, and third dilator tubes 140, 145, 160 may be removed. In one embodiment, rotation of the cannula 130 can gently move the nerve away from the access site while also protecting the nerve as tools and devices may be inserted through the cannula 130. The access cannula 130 can then provide an open lumen 131 through which surgical tools can be introduced to the site of the intervertebral disc 12. As noted above, the positioning of the access cannula 130 protects the exiting nerve (not shown) from coming into contact with any of the surgical tools.

A example of a surgical tool for use through the access cannula is depicted in FIG. 14. The intervertebral implant 80 may be introduced through the access cannula 130, and released once in position. Although a particular intervertebral implant is shown here, one of skill in the art will readily understand that any number of surgical tools may be introduced through the access cannula. For example, surgical tools to be inserted through the access cannula may include, without limitation, discectomy tools, tissue extractors, bone graft insertion tools, rasps, forceps, drills (e.g., trephine), rongeurs, curettes, paddle distractors, mechanical distractors, lasers, automated probes, manual probes, and plasma wands. In one embodiment of use, an opening in the disc annulus can be formed and a portion of the disc can be removed using tools advanced through the access cannula 130. The disc space can be distracted (e.g., using paddle distractors) before and/or after the implant 80 and/or different or additional interbody devices are inserted through the access cannula 130 and placed between the vertebral bodies to maintain spacing. In some embodiments the disc nucleus or portions thereof is removed while leaving the disc annulus. Bone graft and/or other materials such as, for example, bone morphogenetic proteins (BMPs) can be placed between the vertebrae before, while or after positioning the implant. Fusion can then occur between the vertebrae. In some procedures, fusion can be augmented with other fixation devices such as, for example, pedicle screws and rod constructions, transfacet and transpedicle screws, interbody spacers, rods, plates and cages, which can be used to stabilize a pair of vertebral bodies together. For example, in one arrangement, the fusion is augmented by one or more posterior fixation devices (e.g transfacet and transpedicle screws and/or pedicle screws and rods and/or spinous process spacers). In such a manner, the entire fusion procedure can be done from a posterior position and preferably in a minimally invasive (e.g., percutaneous manner). For example, in one embodiment, the above described procedure is used in combination with the transfacet-pedicular implant system sold by Intervention Spine, Inc. under the trade name PERPOS®, such a system is also described in U.S. Pat. Nos. 7,998,176 and 7,824,429, the entirety of which are hereby incorporated by reference herein.

As described in more above, the third dilator tube and the access cannula each have outer surfaces that are substantially rectangular in cross-section. It is understood that the term “rectangular” as used herein also includes a square shape. This stands in contrast to the substantially rounded outer surfaces of the first and second dilator tubes. In some embodiments, the shape and dimensions of the lumen of the access cannula can be configured to receive an intervertebral implant therethrough. In particular, an interveretebral implant having a substantially rectangular cross-section can be passed through the lumen of the access cannula. Due to the substantially rectangular shape, the total cross-sectional size of the lumen can be reduced relative to rounded configurations. For example, in some embodiments the height and width of the lumen can each be reduced by about 2.2 mm relative to a rounded configuration.

In some embodiments, the reduction in these dimensions can allow reduce the need for foraminoplasty and/or can reduce the risk of damaging the traversing nerve root during the procedure. Additionally, the reduced dimensions may aid in accessing particularly tight disc spaces, such as in the L5/S1 region. In some embodiments, the substantially rectangular shape of the third dilator tube can aid the foraminoplasty procedure. The sharper edges, as compared to the rounded configuration, may more readily remove bone to expand the foramen. In some embodiments, the substantially rectangular cross-section of the access cannula lumen advantageously facilitates docking the access cannula within the disc space. The position of the access cannula may thereby be more easily retained, allowing for accurate and precise insertion of intervertebral implants into the disc space. and an outer surface that is substantially rectangular in cross-section. In some embodiments, the outer surface of the third dilator tube is substantially rectangular in cross-section, having a height and a width. In some embodiments, the cross-section may be substantially square, in which case the height and width are approximately equal. The outer surface of the third dilator tube can be centered around the third longitudinal axis. As noted above, the inner longitudinal lumen may also be centered around the third longitudinal axis. The longitudinal lumen of the third dilator tube can have a substantially circular cross-section, while the outer surface of the third dilator tube is substantially rectangular. As with the outer surface of the third dilator tube, the lumen of the access cannula can have a substantially rectangular cross-section, and can have a width and a height. In some embodiments, the outer surface of the third dilator tube and the inner lumen of the access cannula are both substantially rectangular in cross-section. As such, the third dilator tube, in such a configuration, cannot be rotated with respect to the access cannula. The access cannula can slide proximally and distally relative to the third dilator tube, but their relative rotational orientation may remain fixed. Even while fixed with respect to one another, however, both the access cannula and the third dilator tube may, together, rotate with respect to the second dilator tube and/or the first dilator tube. beveled or tapered shape, in which the is a partial rectangle or U-shaped surface.

### Implant

With respect to the implant 80 described above, the implant 80 can comprise any of a variety of types of interbody devices configured to be placed between vertebral bodies. The implant 80 can be formed from a metal (e.g., titanium) or a non-metal material such as plastics, PEEK™, polymers, and rubbers. Further, the implant components can be made of combinations of non metal materials (e.g., PEEK™, polymers) and metals. The implant 80 can be configured with a fixed or substantially fixed height, length and width as shown, for example, in the embodiment of FIG. 14. In other embodiments, the implant can be configured to be expandable along one or more directions. For example, in certain embodiments the height of the implant can be expanded once the device advanced through the access cannula and positioned between vertebral bodies (e.g., within the disc space within the annulus).

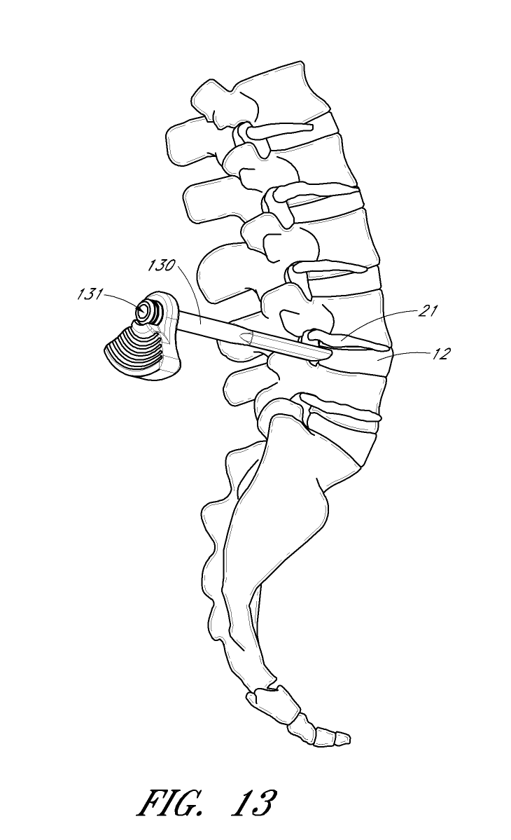

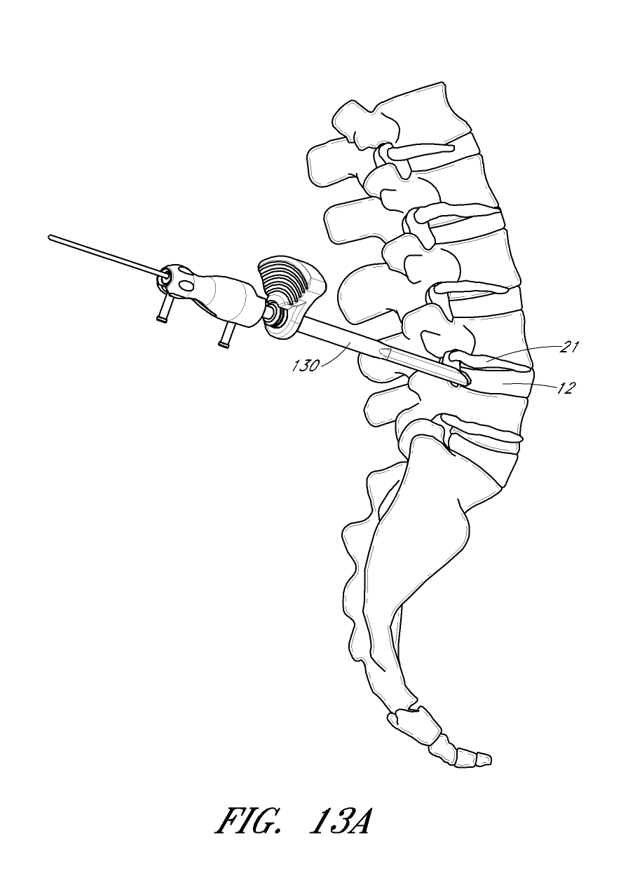

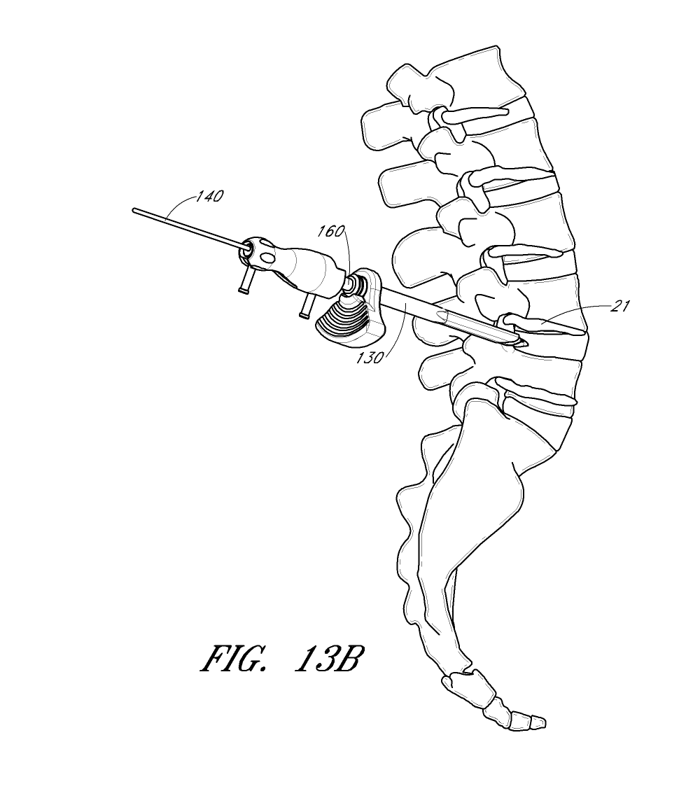

Additional detail of one embodiment of such an expandable implant can be found in FIGS. 15A-25. As shown, in FIGS. 15A-B, in the illustrated embodiments, the implant 200 can be configured such that proximal and distal wedge members 206, 208 are interlinked with upper and lower body portions 202, 204. The upper and lower body portions 202, 204 can include slots (slot 220 is shown in FIG. 15A, and slots 220, 222 are shown in FIG. 15B; the configuration of such an embodiment of the upper and lower body portions 202, 204 is also shown in FIGS. 15A-16B, discussed below). In such an embodiment, the proximal and distal wedge members 206, 208 can include at least one guide member (an upper guide member 230 of the proximal wedge member 206 is shown in FIG. 15A and an upper guide member 232 of the distal wedge member 208 is shown in FIG. 17) that at least partially extends into a respective slot of the upper and lower body portions. The arrangement of the slots and the guide members can enhance the structural stability and alignment of the implant 200.

In addition, it is contemplated that some embodiments of the implant 200 can be configured such that the upper and lower body portions 202, 204 each include side portions (shown as upper side portion 240 of the upper body portion 202 and lower side portion 242 of the lower body portion 204) that project therefrom and facilitate the alignment, interconnection, and stability of the components of the implant 200. FIG. 15B is a perspective view of the implant 200 wherein the implant 200 is in the expanded state. The upper and lower side portions 240, 242 can be configured to have complementary structures that enable the upper and lower body portions 202, 204 to move in a vertical direction. Further, the complementary structures can ensure that the proximal ends of the upper and lower body portions 202, 204 generally maintain spacing equal to that of the distal ends of the upper and lower body portions 202, 204. The complementary structures are discussed further below with regard to FIGS. 16-20B.

Furthermore, as described further below, the complementary structures can also include motion limiting portions that prevent expansion of the implant beyond a certain height. This feature can also tend to ensure that the implant is stable and does not disassemble during use.

In some embodiments, the actuator shaft 210 can facilitate expansion of the implant 200 through rotation, longitudinal contract of the pin, or other mechanisms. The actuator shaft 210 can include threads that threadably engage at least one of the proximal and distal wedge members 206, 208. The actuator shaft 210 can also facilitate expansion through longitudinal contraction of the actuator shaft as proximal and distal collars disposed on inner and outer sleeves move closer to each other to in turn move the proximal and distal wedge members closer together. It is contemplated that in other embodiments, at least a portion of the actuator shaft can be axially fixed relative to one of the proximal and distal wedge members 206, 208 with the actuator shaft being operative to move the other one of the proximal and distal wedge members 206, 208 via rotational movement or longitudinal contraction of the pin.

Further, in embodiments wherein the actuator shaft 210 is threaded, it is contemplated that the actuator shaft 210 can be configured to bring the proximal and distal wedge members closer together at different rates. In such embodiments, the implant 200 could be expanded to a V-configuration or wedged shape. For example, the actuator shaft 210 can comprise a variable pitch thread that causes longitudinal advancement of the distal and proximal wedge members at different rates. The advancement of one of the wedge members at a faster rate than the other could cause one end of the implant to expand more rapidly and therefore have a different height than the other end. Such a configuration can be advantageous depending on the intervertebral geometry and circumstantial needs.

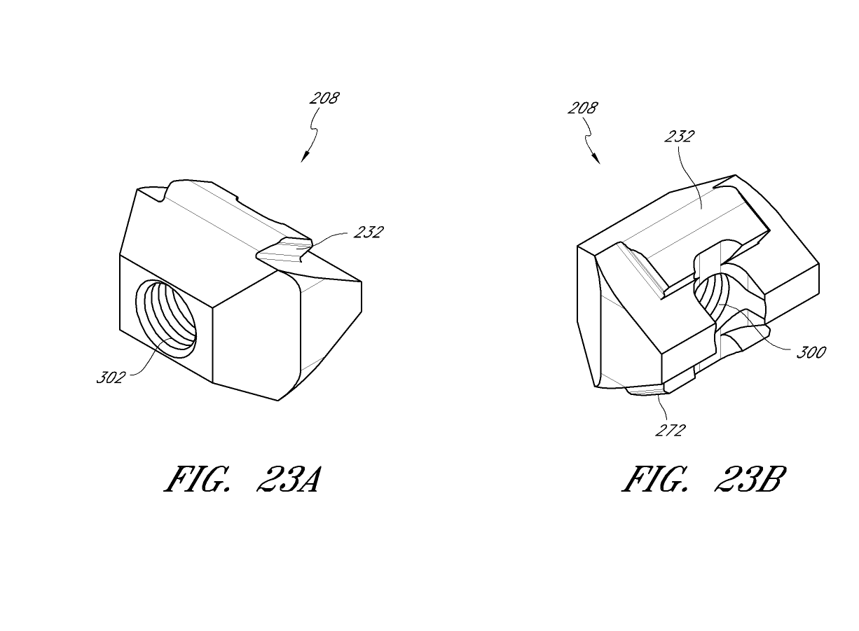

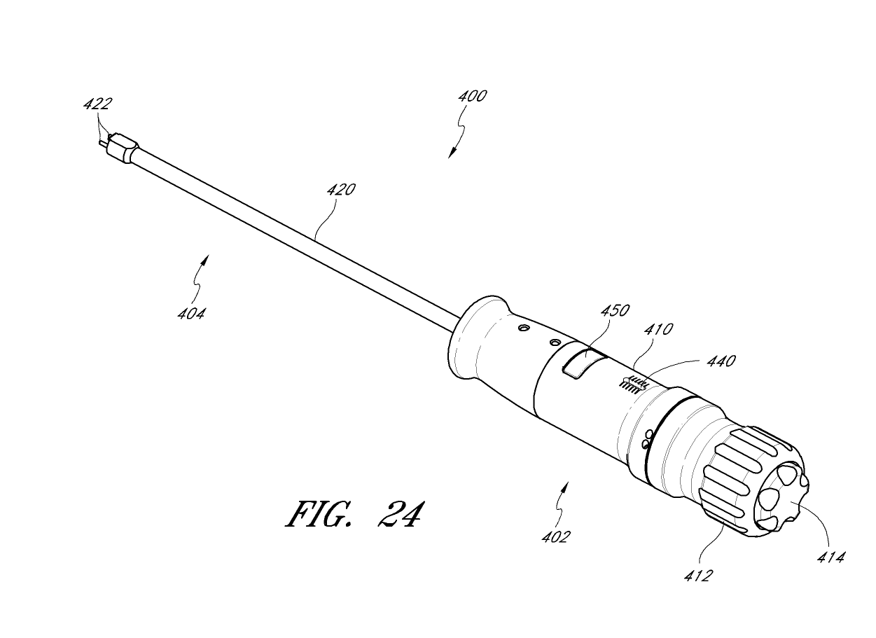

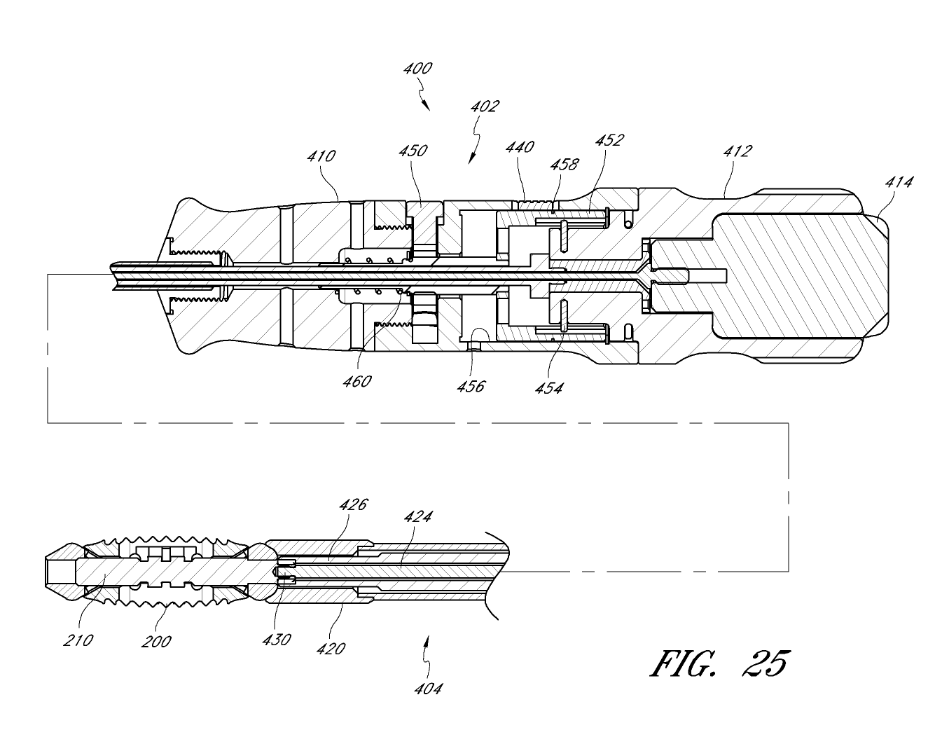

In other embodiments, the implant 200 can be configured to include anti-torque structures 250. The anti-torque structures 250 can interact with at least a portion of a deployment tool during deployment of the implant to ensure that the implant maintains its desired orientation (see FIGS. 24-25 and related discussion). For example, when the implant 200 is being deployed and a rotational force is exerted on the actuator shaft 210, the anti-torque structures 250 can be engaged by a non-rotating structure of the deployment tool to maintain the rotational orientation of the implant 200 while the actuator shaft 210 is rotated. The anti-torque structures 250 can comprise one or more inwardly extending holes or indentations on the proximal wedge member 206, which are shown as a pair of holes in FIGS. 15A-B. However, the anti-torque structures 250 can also comprise one or more outwardly extending structures.

According to yet other embodiments, the implant 200 can be configured to include one or more apertures 252 to facilitate osseointegration of the implant 200 within the intervertebral space. As mentioned above, the implant 200 may contain one or more bioactive substances, such as antibiotics, chemotherapeutic substances, angiogenic growth factors, substances for accelerating the healing of the wound, growth hormones, antithrombogenic agents, bone growth accelerators or agents, and the like. Indeed, various biologics can be used with the implant 200 and can be inserted into the disc space or inserted along with the implant 200. The apertures 252 can facilitate circulation and bone growth throughout the intervertebral space and through the implant 200. In such implementations, the apertures 252 can thereby allow bone growth through the implant 200 and integration of the implant 200 with the surrounding materials.

FIG. 16 is a bottom view of the implant 200 shown in FIG. 15A. As shown therein, the implant 200 can comprise one or more protrusions 260 on a bottom surface 262 of the lower body portion 204. Although not shown in this Figure, the upper body portion 204 can also define a top surface having one or more protrusions thereon. The protrusions 260 can allow the implant 200 to engage the adjacent vertebrae when the implant 200 is expanded to ensure that the implant 200 maintains a desired position in the intervertebral space.

The protrusions 260 can be configured in various patterns. As shown, the protrusions 260 can be formed from grooves extending widthwise along the bottom surface 262 of the implant 200 (also shown extending from a top surface 264 of the upper body portion 202 of the implant 200). The protrusions 260 can become increasingly narrow and pointed toward their apex. However, it is contemplated that the protrusions 260 can be one or more raised points, cross-wise ridges, or the like.

FIG. 16 also illustrates a bottom view of the profile of an embodiment of the upper side portion 240 and the profile of the lower side portion 242. As mentioned above, the upper and lower side portions 240, 242 can each include complementary structures to facilitate the alignment, interconnection, and stability of the components of the implant 200. FIG. 16 also shows that in some embodiments, having a pair of each of upper and lower side portions 240, 242 can ensure that the upper and lower body portions 202, 204 do not translate relative to each other, thus further ensuring the stability of the implant 200.

As illustrated in FIG. 16, the upper side portion 240 can comprise a groove 266 and the lower side portion can comprise a rib 268 configured to generally mate with the groove 266. The groove 266 and rib 268 can ensure that the axial position of the upper body portion 202 is maintained generally constant relative to the lower body portion 204. Further, in this embodiment, the grooves 266 and rib 268 can also ensure that the proximal ends of the upper and lower body portions 202, 204 generally maintain spacing equal to that of the distal ends of the upper and lower body portions 202, 204. This configuration is also illustratively shown in FIG. 17.

Referring again to FIG. 16, the implant 200 is illustrated in the unexpanded state with each of the respective slots 222 of the lower body portion 204 and lower guide members 270, 272 of the respective ones of the proximal and distal wedge members 206, 208. In some embodiments, as shown in FIGS. 15A-16 and 18-20B, the slots and guide members can be configured to incorporate a generally dovetail shape. Thus, once a given guide member is slid into engagement with a slot, the guide member can only slide longitudinally within the slot and not vertically from the slot. This arrangement can ensure that the proximal and distal wedge members 206, 208 are securely engaged with the upper and lower body portions 202, 204.

Furthermore, in FIG. 17, a side view of the embodiment of the implant 200 in the expanded state illustrates the angular relationship of the proximal and distal wedge members 206, 208 and the upper and lower body portions 202, 204. As mentioned above, the dovetail shape of the slots and guide members ensures that for each given slot and guide member, a given wedge member is generally interlocked with the give slot to only provide one degree of freedom of movement of the guide member, and thus the wedge member, in the longitudinal direction of the given slot.

Accordingly, in such an embodiment, the wedge members 206, 208 may not be separable from the implant when the implant 200 is in the unexpanded state (as shown in FIG. 15A) due to the geometric constraints of the angular orientation of the slots and guide members with the actuator shaft inhibiting longitudinal relative movement of the wedge members 206, 208 relative to the upper and lower body portions 202, 204. Such a configuration ensures that the implant 200 is stable and structurally sound when in the unexpanded state or during expansion thereof, thus facilitating insertion and deployment of the implant 200.

Such an embodiment of the implant 200 can therefore be assembled by placing or engaging the wedge members 206, 208 with the actuator shaft 210, moving the wedge members 206, 208 axially together, and inserting the upper guide members 230, 232 into the slots 220 of the upper body portion 202 and the lower guide members 270, 272 into the slots 222 of the lower body portion 204. The wedge members 206, 208 can then be moved apart, which movement can cause the guide members and slots to engage and bring the upper and lower body portions toward each other. The implant 200 can then be prepared for insertion and deployment by reducing the implant 200 to the unexpanded state.

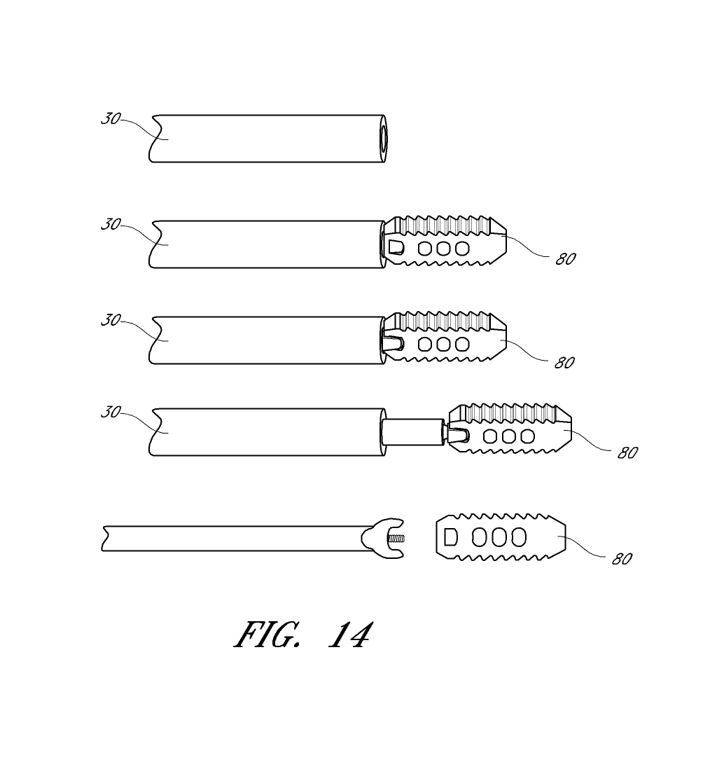

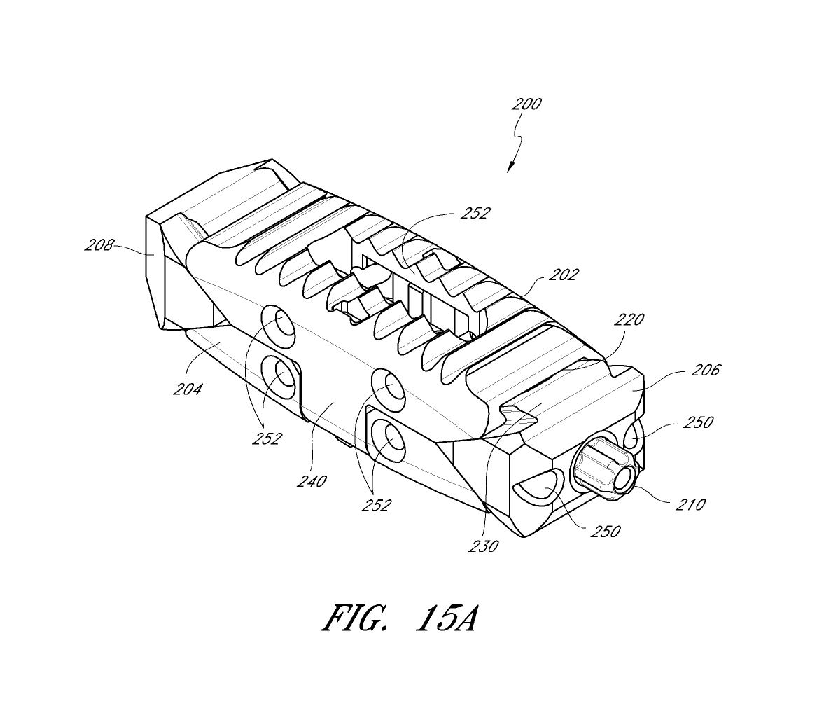

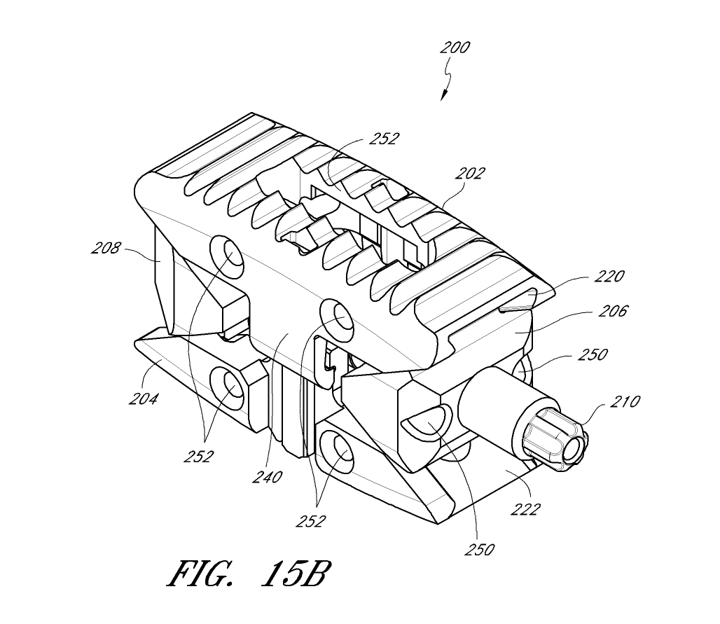

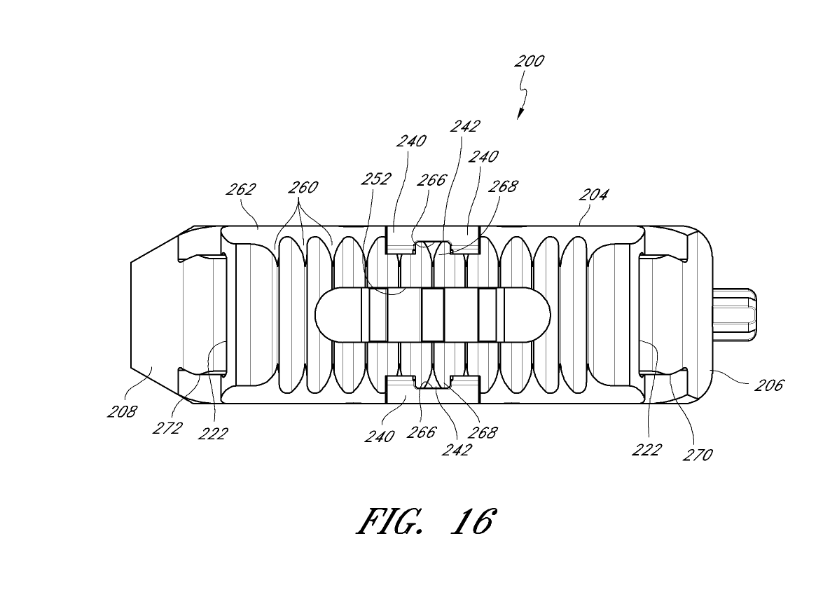

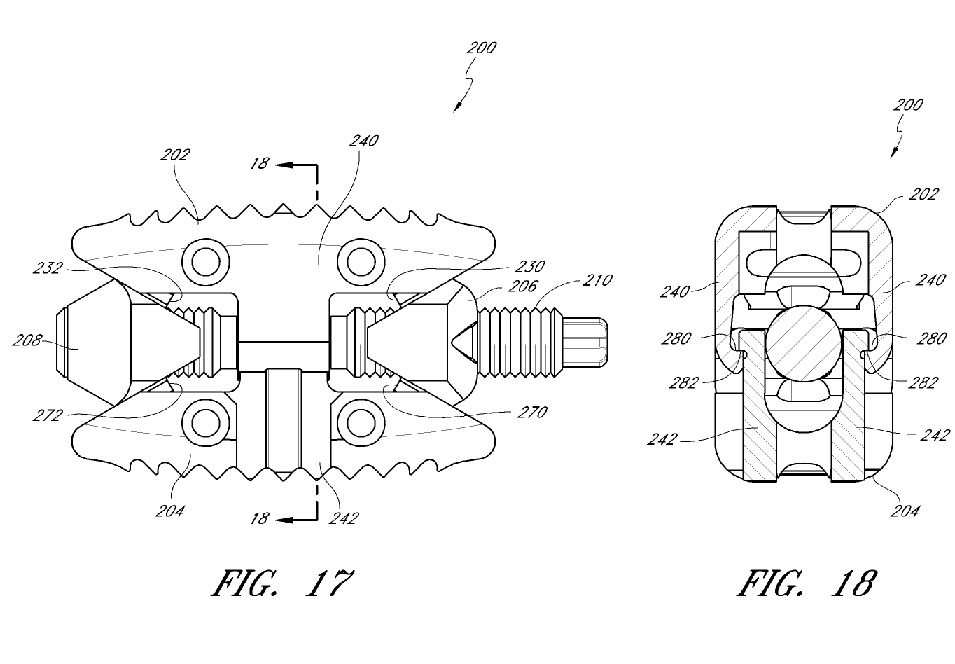

During assembly of the implant 200, the upper and lower body portions 202, 204 can be configured to snap together to limit expansion of the implant 200. For example, the upper and lower side portions 240, 242 can comprise upper and lower motion-limiting structures 280, 282, as shown in the cross-sectional view of FIG. 18. After the wedge members 206, 208 are engaged with the upper and lower body portions 202, 204 and axially separated to bring the upper and lower body portions 202, 204 together, the upper motion-limiting structure 280 can engage the lower motion-limiting structure 282. This engagement can occur due to deflection of at least one of the upper and lower side portions 240, 242. However, the motion-limiting structures 280, 282 preferably comprise interlocking lips or shoulders to engage one another when the implant 200 has reached maximum expansion. Accordingly, after the wedge members 206, 208 are assembled with the upper and lower body portions 202, 204, these components can be securely interconnected to thereby form a stable implant 200.

Referring again to FIG. 17, the implant 200 can define generally convex top and bottom surfaces 264, 262. In modified embodiments, the shape can be modified.

FIGS. 19A-B illustrate perspective views of the lower body portion 204 of the implant 200, according to an embodiment. These Figures provide additional clarity as to the configuration of the slots 222, the lower side portions 242, and the lower motion-limiting members 282 of the lower body portion 204. Similarly, FIGS. 20A-B illustrate perspective views of the upper body portion 202 of the implant 200, according to an embodiment. These Figures provide additional clarity as to the configuration of the slots 220, the upper side portions 240, and the upper motion-limiting members 280 of the upper body portion 202. Additionally, the upper and lower body portions 202, 204 can also define a central receptacle 290 wherein the actuator shaft can be received. Further, as mentioned above, the upper and lower body portions 202, 204 can define one or more apertures 252 to facilitate osseointegration.

FIG. 21 is a perspective view of an actuator shaft 210 of the implant 200 shown in FIG. 15. In this embodiment, the actuator shaft 210 can be a single, continuous component having threads 294 disposed thereon for engaging the proximal and distal wedge members 206, 208. The threads can be configured to be left hand threads at a distal end of the actuator shaft 210 and right hand threads at a proximal other end of the actuator shaft for engaging the respective ones of the distal and proximal wedge members 208, 206. Accordingly, upon rotation of the actuator shaft 210, the wedge members 206, 208 can be caused to move toward or away from each other to facilitate expansion or contraction of the implant 200. Further, as noted above, although this embodiment is described and illustrated as having the actuator shaft 210 with threads 294.

In accordance with an embodiment, the actuator shaft 210 can also comprise a tool engagement section 296 and a proximal engagement section 298. The tool engagement section 296 can be configured as a to be engaged by a tool, as described further below. The tool engagement section 296 can be shaped as a polygon, such as a hex shape. As shown, the tool engagement section 296 is star shaped and includes six points, which configuration tends to facilitate the transfer of torque to the actuator shaft 210 from the tool. Other shapes and configurations can also be used.

Furthermore, the proximal engagement section 298 of the actuator shaft 210 can comprise a threaded aperture. The threaded aperture can be used to engage a portion of the tool for temporarily connecting the tool to the implant 200. It is also contemplated that the proximal engagement section 298 can also engage with the tool via a snap or press fit.

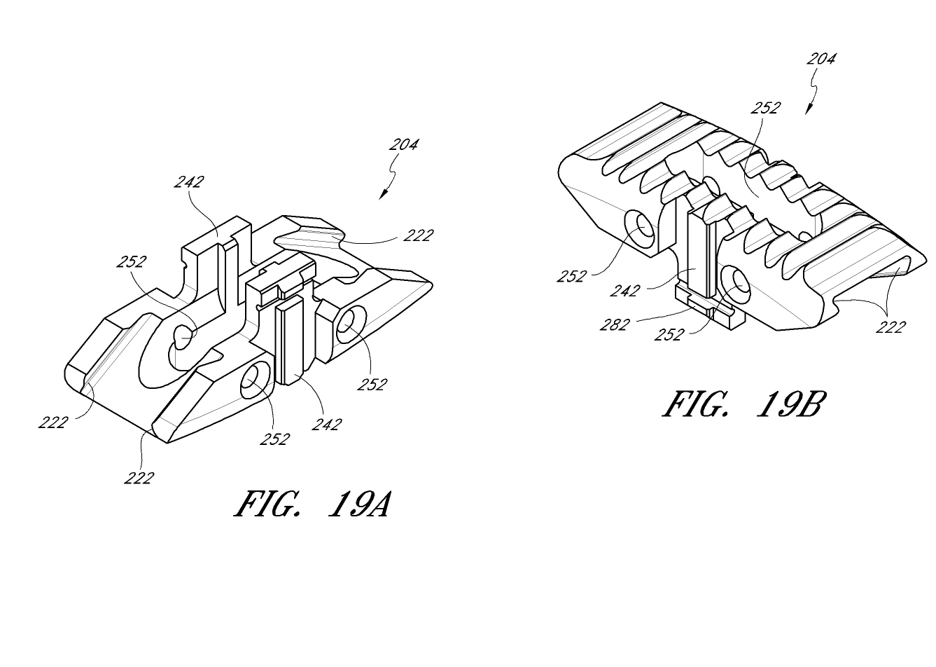

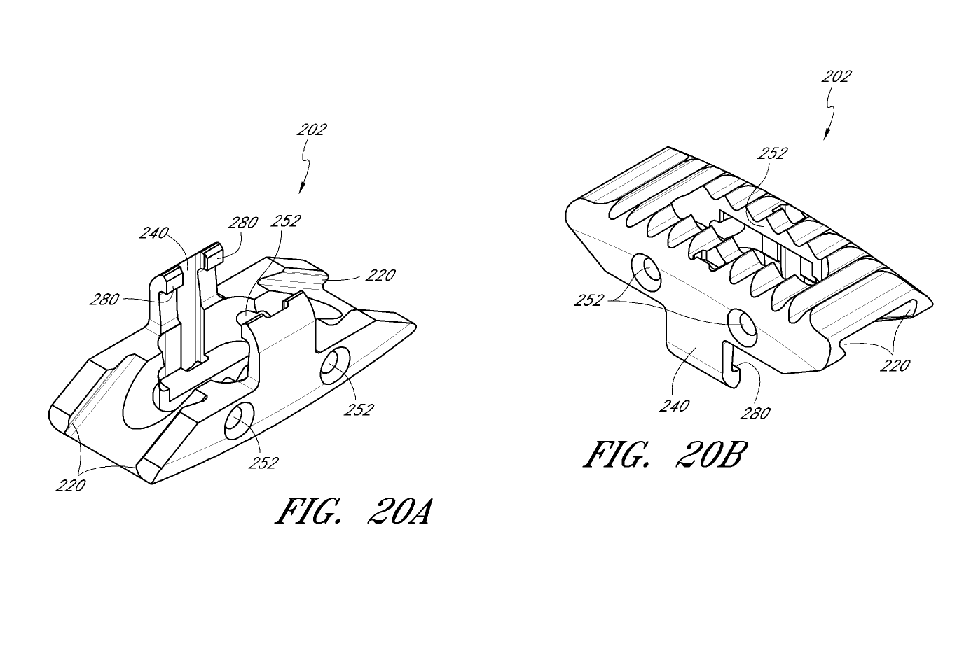

FIG. 22A-B illustrate perspective views of the proximal wedge member 206 of the implant 200. As described above, the proximal wedge member can include one or more anti-torque structures 250. Further, the guide members 230, 270 are also illustrated. The proximal wedge member 206 can comprise a central aperture 300 wherethrough an actuator shaft can be received. When actuator shaft 210 is used in an embodiment, the central aperture 300 can be threaded to correspond to the threads 294 of the actuator shaft 210. In other embodiments, the actuator shaft can engage other portions of the wedge member 206 for causing expansion or contraction thereof.

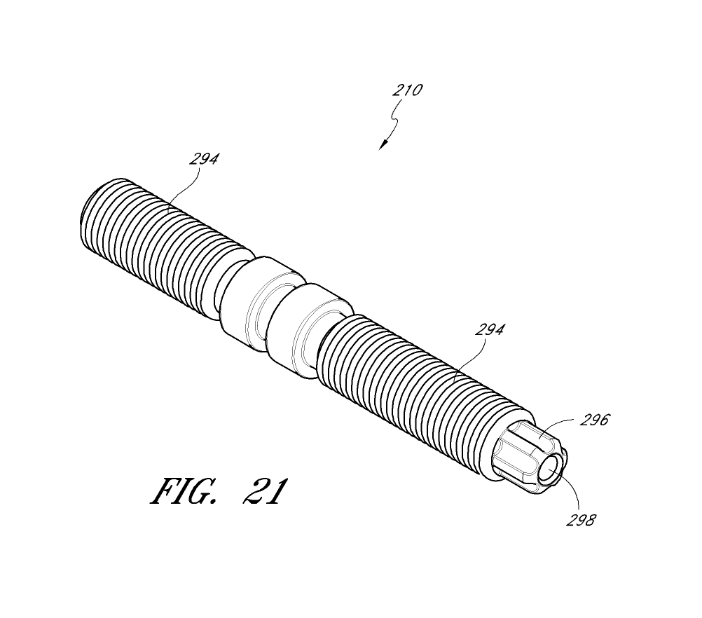

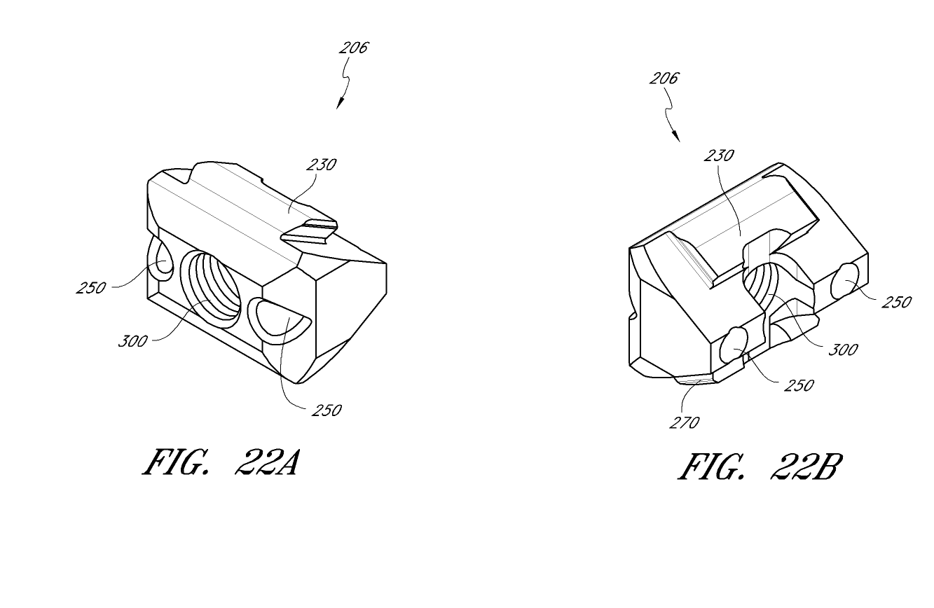

FIG. 23A-B illustrate perspective views of the distal wedge member 208 of the implant 200. As similarly discussed above with respect to the proximal wedge member 206, the guide members 232, 272 and a central aperture 302 of the proximal wedge member 206 are illustrated. The central aperture 302 can be configured to receive an actuator shaft therethrough. When actuator shaft 210 is used in an embodiment, the central aperture 302 can be threaded to correspond to the threads 294 of the actuator shaft 210. In other embodiments, the actuator shaft can engage other portions of the wedge member 208 for causing expansion or contraction thereof.

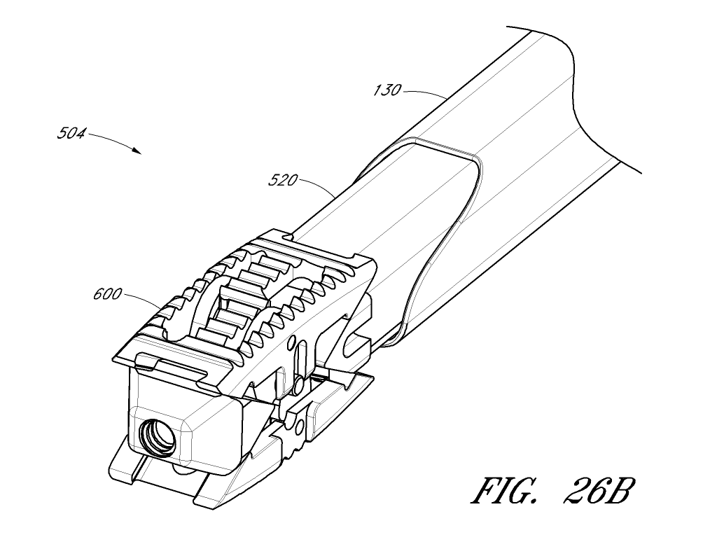

Referring now to FIG. 27, there is illustrated a perspective view of a deployment tool 400 according to another embodiment. The tool 400 can comprise a handle section 402 and a distal engagement section 404. The handle portion 402 can be configured to be held by a user and can comprise various features to facilitate implantation and deployment of the implant.

According to an embodiment, the handle section 402 can comprise a fixed portion 410, and one or more rotatable portions, such as the rotatable deployment portion 412 and the rotatable tethering portion 414. In such an embodiment, the tethering portion 414 can be used to attach the implant to the tool 400 prior to insertion and deployment. The deployment portion 412 can be used to actuate the implant and rotate the actuator shaft thereof for expanding the implant. Then, after the implant is expanded and properly placed, the tethering portion 414 can again be used to untether or decouple the implant from the tool 400.

Further, the distal engagement section 404 can comprise a fixed portion 420, an anti-torque component 422, a tethering rod (element 424 shown in FIG. 25), and a shaft actuator rod (element 426 shown in FIG. 21) to facilitate engagement with and actuation of the implant 200. The anti-torque component 422 can be coupled to the fixed portion 420. As described above with reference to FIGS. 15A-B, in an embodiment, the implant 200 can comprise one or more anti-torque structures 250. The anti-torque component 422 can comprise one or more protrusions that engage the anti-torque structures 250 to prevent movement of the implant 200 when a rotational force is applied to the actuator shaft 210 via the tool 400. As illustrated, the anti-torque component 422 can comprise a pair of pins that extend from a distal end of the tool 400. However, it is contemplated that the implant 200 and tool 400 can be variously configured such that the anti-torque structures 250 and the anti-torque component 422 interconnect to prevent a torque being transferred to the implant 200. The generation of the rotational force will be explained in greater detail below with reference to FIG. 25, which is a side-cross sectional view of the tool 400 illustrating the interrelationship of the components of the handle section 402 and the distal engagement section 404.

For example, as illustrated in FIG. 25, the fixed portion 410 of the handle section 402 can be interconnected with the fixed portion 420 of the distal engagement section 404. The distal engagement section 404 can be configured with the deployment portion 412 being coupled with the shaft actuator rod 426 and the tethering portion 414 being coupled with the tethering rod 424. Although these portions can be coupled to each other respectively, they can move independently of each other and independently of the fixed portions. Thus, while holding the fixed portion 410 of the handle section 402, the deployment portion 412 and the tethering portion 414 can be moved to selectively expand or contract the implant or to attach the implant to the tool, respectively. In the illustrated embodiment, these portions 412, 414 can be rotated to cause rotation of an actuator shaft 210 of an implant 200 engaged with the tool 400.

As shown in FIG. 25, the tether rod 424 can comprise a distal engagement member 430 being configured to engage a proximal end of the actuator shaft 210 of the implant 200 for rotating the actuator shaft 210 to thereby expand the implant from an unexpanded state to and expanded state. The tether rod 424 can be configured with the distal engagement member 430 being a threaded distal section of the rod 424 that can be threadably coupled to an interior threaded portion of the actuator shaft 210. As mentioned above, the anti-torque component 422 of the

In some embodiments, the tool 400 can be prepared for a single-use and can be packaged with an implant preloaded onto the tool 400. This arrangement can facilitate the use of the implant and also provide a sterile implant and tool for an operation. Thus, the tool 400 can be disposable after use in deploying the implant.

Referring again to FIG. 24, an embodiment of the tool 400 can also comprise an expansion indicator gauge 440 and a reset button 450. The expansion indicator gauge 440 can be configured to provide a visual indication corresponding to the expansion of the implant 200. For example, the gauge 440 can illustrate an exact height of the implant 200 as it is expanded or the amount of expansion. As shown in FIG. 25, the tool 400 can comprise a centrally disposed slider element 452 that can be in threaded engagement with a thread component 454 coupled to the deployment portion 412.

In an embodiment, the slider element 452 and an internal cavity 456 of the tool can be configured such that the slider element 452 is provided only translational movement in the longitudinal direction of the tool 400. Accordingly, as the deployment portion 412 is rotated, the thread component 454 is also rotated. In such an embodiment, as the thread component 454 rotates and is in engagement with the slider component 452, the slider element 452 can be incrementally moved from an initial position within the cavity 456 in response to the rotation of the deployment portion 412. An indicator 458 can thus be longitudinally moved and viewed to allow the gauge 440 to visually indicate the expansion and/or height of the implant 200. In such an embodiment, the gauge 440 can comprises a transparent window through which the indicator 458 on the slider element 452 can be seen. In the illustrated embodiment, the indicator 458 can be a marking on an exterior surface of the slider element 452.

In embodiments where the tool 400 can be reused, the reset button 450 can be utilized to zero out the gauge 440 to a pre-expansion setting. In such an embodiment, the slider element 452 can be spring-loaded, as shown with the spring 460 in FIG. 25. The reset button 450 can disengage the slider element 452 and the thread component 454 to allow the slider element 452 to be forced back to the initial position.

Additional details and embodiments of an expandable implant can be found in U.S. Patent Application No 2008/0140207, filed Dec. 7, 2007 as U.S. patent application Ser. No. 11/952,900, and U.S. patent application Ser. No. 13/789,507, filed Mar. 7, 2013. The entirety of each of these applications is hereby incorporated by reference herein.

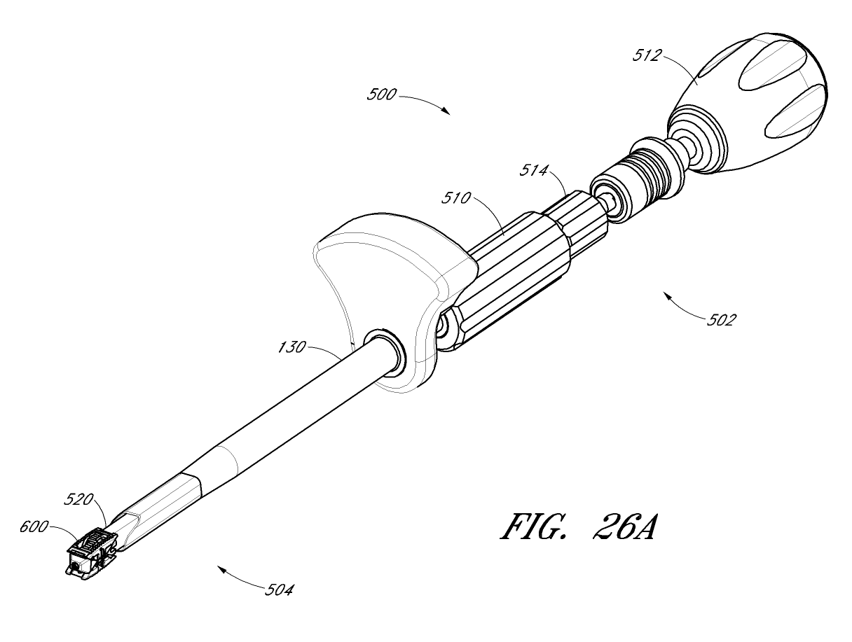

FIG. 26A illustrates a perspective view of another embodiment of a deployment tool 500 engaged with an access cannula 130 (described above with respect to FIGS. 4A-12C). FIGS. 26B and 26C illustrate enlarged perspective views of the distal end of the deployment tool 500, with the implant engaged in FIG. 26B, and removed in FIG. 26C. Similar to the deployment tool of FIGS. 3024 and 3125, the deployment tool 300 comprises a handle section 502 and a distal engagement section 504. The tool 300 includes a tethering portion 514 coupled to a tethering rod (not shown) that can be used to attach the implant 600 to the tool 500 prior to insertion and deployment. The tool 500 also includes a deployment portion 512 coupled to a shaft actuator rod with a distal engagement member 530 at its distal end. The deployment portion 512 can be used to actuate the implant 600 and rotate the actuator shaft thereof for expanding the implant 600. After the implant 600 is expanded and properly placed, the tethering portion 514 can again be used to untether or decouple the implant from the tool 500. Fixed portion 510 of the handle portion 502 is coupled to the fixed portion 520 of the distal engagement section 504. In the illustrated embodiment, the shaft actuator rod of the deployment portion 512 is positioned within the tethering rod of the tethering portion 514, in contrast to the embodiment described above with respect to FIGS. 24-25, in which the tethering rod is positioned within the shaft actuator rod. To accommodate this configuration, the implant 600 may differ from that described above with respect to FIGS. 15A-23B such that the proximal engagement section is larger than, and surrounds, the tool engagement section.

The fixed portion 520 comprises anti-torque elements 522, which are configured to engage the implant 600 as described above with respect to FIGS. 24-25. However, unlike the substantially cylindrical fixed portion 420 described above, in the illustrated embodiment the fixed portion 520 has a substantially rectangular cross-section, configured for insertion through the rectangular lumen of the access cannula 130. In other embodiments, the fixed portion 520 need not be rectangularly shaped, but rather may assume a cylindrical, elliptical, polygonal, or other shape, so long as the fixed portion 520 is shaped and dimensioned such that it can be inserted through the lumen of the access cannula 130. The implant 600, as illustrated, has a substantially rectangular cross-section, which can be similar in size and shape to the cross-section of the lumen of the access cannula 130. In this configuration, the lumen of the access cannula 130 can assume the smallest possible size while still allowing for the implant 600 to be inserted and deployed therethrough. The cross-sectional size of the access cannula 130 can therefore be significantly reduced compared to cylindrical embodiments.

As noted above, the reduction in the dimensions of the access cannula 130 can reduce the need for foraminoplasty and/or can reduce the risk of damaging the traversing nerve root during the procedure. Additionally, the reduced dimensions may aid in accessing particularly tight disc spaces, such as in the L5/S1 region. In some embodiments, the substantially rectangular shape of the third dilator tube 160 can aid the foraminoplasty procedure. The sharper edges, as compared to the rounded configuration, may more readily remove bone to expand the foramen. In some embodiments, the substantially rectangular cross-section of the access cannula lumen advantageously facilitates docking the access cannula within the disc space. The position of the access cannula may thereby be more easily retained, allowing for accurate and precise insertion of intervertebral implants into the disc space.

Another example of a surgical tool for use through the access cannula is a bone rasp. A rasp tool can be configured to be inserted through the access cannula into the intervertebral disc space. The rasping tool can then be used to abrade or file the inferior surface of the superior vertebrae and/or the superior surface of the inferior vertebrae. The rasping tool may comprise an elongated body and a scraping component. A handle may be proximally attached to the elongated body. The rasping tool includes an open sleeve within which the elongate body is slidably received. This configuration may permit the elongated body 810 and scraping component to slide relative to the open sleeve.

The entire assembly, including the elongate body, open sleeve, and scraping component can be dimensioned such that the rasping tool can slide longitudinally within the access cannula. In use, the rasp tool may be inserted through the access cannula until it reaches the intervertebral disc space. Using the handle, a physician may slide the elongate body and scraping component backward and forward, while the open sleeve remains stationary relative to the access cannula. In other embodiments, the open sleeve is omitted, and the elongate body is inserted directly into the access cannula, and is dimensioned to slidably move within it. In certain embodiments, the elongate body may freely rotate within the open sleeve, or within the access cannula, in order to permit the physician to rasp a surface at any desired angle. In other embodiments, the orientation of the elongate body may be fixed, such that rasping is only permitted along a predetermined angle relative to the access cannula.

In certain embodiments, the rasping tool may be expandable. For example, a rasp tool can be configured to define an unexpanded configuration. When the tool is initially inserted into the working sleeve, the tool can be positioned in the unexpanded configuration. After the tool is advanced into the intervertebral disc, the tool can be expanded to the expanded configuration.

The tool can comprise an elongated body and one or more scraping components. The scraping components can each comprise an outer surface that is configured to scrape or create friction against the disc. For example, the outer surfaces can be generally arcuate and provide an abrasive force when in contact with the interior portion of the disc. In particular, it is contemplated that once the tool is expanded, the scraping components can rasp or scrape against the vertebral end plates of the disc from within an interior cavity formed in the disc. In this manner, the tool can prepare the surfaces of the interior of the disc by removing any additional gelatinous nucleus material, as well as smoothing out the general contours of the interior surfaces of the disc. The rasping may thereby prepare the vertebral endplates for fit with the implant as well as to promote bony fusion between the vertebrae and the implant. Due to the preparation of the interior surfaces of the disc, the placement and deployment of the implant will tend to be more effective.

It is contemplated that the tool can comprise an expansion mechanism that allows the scraping components to move from the unexpanded to the expanded configuration. For example, the tool can be configured such that the scraping components expand from an outer dimension or height of approximately 9 mm to approximately 13 mm. In this regard, the expansion mechanism can be configured similarly to the expansion mechanisms of the implants disclosed herein, the disclosure for which is incorporated here and will not be repeated.

Further, it is contemplated that the scraping components can comprise one or more surface structures, such as spikes, blades, apertures, etc. that allow the scraping components 812 to not only provide an abrasive force, but that also allowed the scraping components 812 to remove material from the disc. In this regard, as in any of the implementations of the method, a cleaning tool can be used to remove loosened, scraped, or dislodged disc material. Accordingly, in various embodiments of the methods disclosed herein, and embodiment of the tool 800 can be used to prepare the implant site (the interior cavity of the disc) to optimize the engagement of the implant with the surfaces of the interior of the disc (the vertebral end plates).

After the implant site has been prepared, the implant can be advanced through the second working sleeve into the disc cavity. Once positioned, the implant can be expanded to its expanded configuration. For example, the implant can be expanded from approximately 9 mm to approximately 12.5 mm. The surgeon can adjust the height and position of the implant as required. Additionally, other materials or implants can then be installed prior to the removal of the second working sleeve and closure of the implant site.

### Graft Delivery Device

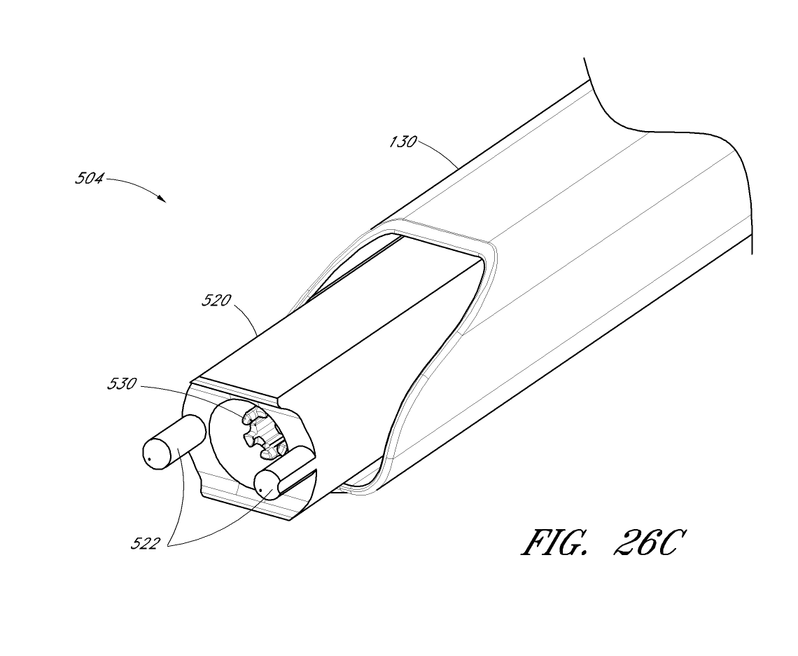

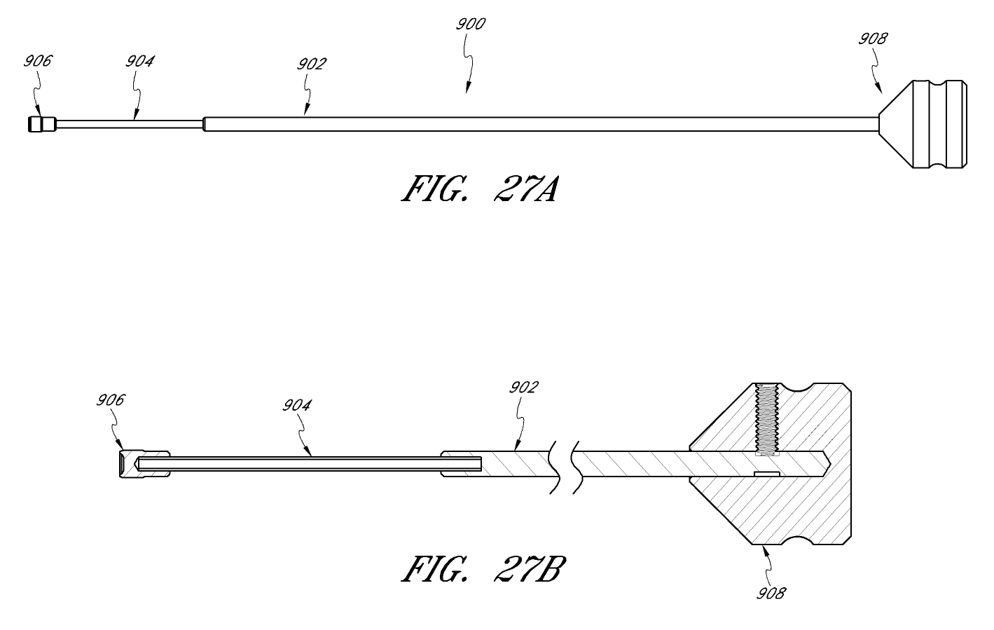

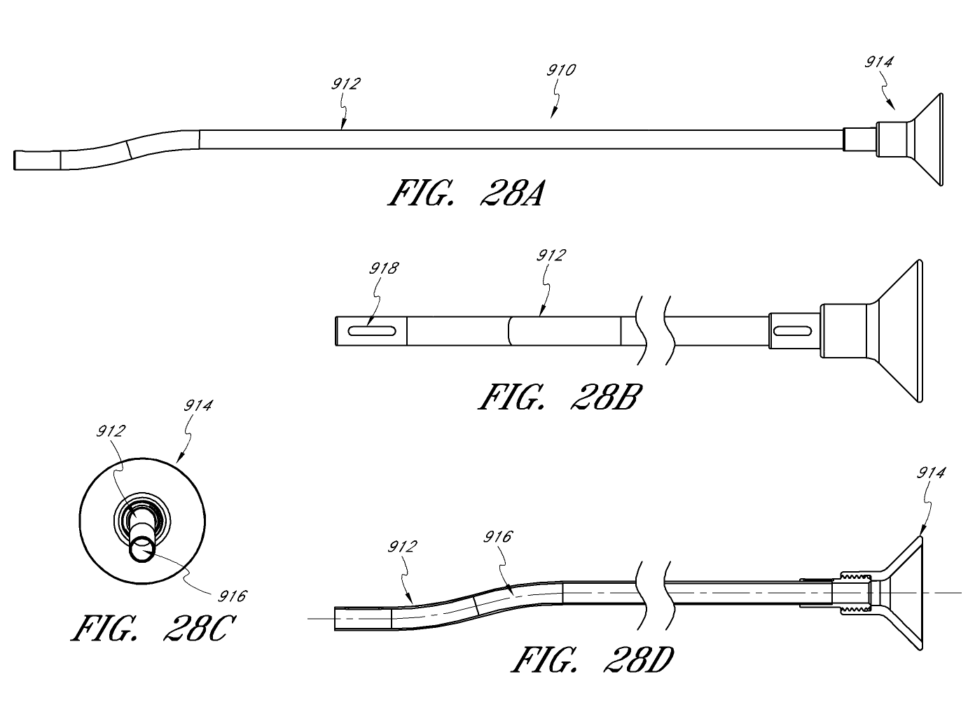

With reference now to FIGS. 27A to 28D, a bone graft delivery device is disclosed which may be inserted through the access cannula for use in the intervertebral space. For example, the bone graft material can be inserted into the intervertebral disc space in order to promote rapid fixation between the adjacent vertebrae. The bone graft material may be inserted before insertion of an intervertebral implant. Alternatively, the bone graft material may be inserted following insertion of the intervertebral implant. In some implementations, bone graft material is delivered both prior to and following insertion of the intervertebral implant. Bone graft material may be autologous, allograft, xenograft, or synthetic. In addition to bone graft material, other materials may be introduced to the treatment site, as desired. For example, bone morphogenetic proteins may be introduced with a carrier medium, such as a collagen, through use of the disclosed delivery device.

FIGS. 27A and 27B show a plunger assembly 900. The plunger assembly 900 includes an elongate shaft 902. In some embodiments, the shaft 902 is substantially rigid. The plunger assembly 900 includes a distal tip 906, which is connected to the elongate shaft 902 by a flexible member 904. A plunger knob 908 is positioned at the proximal end of the plunger assembly 900.

FIGS. 28A-D show a funnel assembly 910. The funnel assembly 910 includes a bent shaft 912. The bent shaft 912 may be substantially straight along the majority of its length, with a bend positioned nearer the distal portion of the bent shaft 912. In other embodiments, a plurality of bends may be included in the bent shaft 912. The particular orientation of the bend may be adjusted to provide for improved access to the intervertebral disc space when the funnel assembly is inserted through the access cannula. A receptacle 914 is located at the proximal end of the funnel assembly 910.

The bent shaft 912 includes a central lumen 916 which runs from the opening of the receptacle at the proximal end to the distal opening of the funnel assembly 910. The plunger assembly 900 is configured to be slidably received within the funnel assembly 910. Accordingly, the dimensions of the distal tip 906, flexible member 904 and the elongate shaft 902 are such that they may slide into the opening at the receptacle 914 of the funnel assembly 910. As the plunger assembly 900 is advanced through the lumen 916 of the funnel assembly 910, the distal tip 906 may reach the bent portion of the bent shaft 912. Due to the pliable nature of flexible member 904, the distal tip 906 may be advanced along lumen 916 through the curve in bent shaft 912. The plunger knob 908 may be configured to be mated with the receptacle 914, such that when the plunger assembly 900 is fully advanced into the funnel assembly 910, the plunger knob 908 contacts the receptacle 914. As shown, the receptacle 914 has a hollow conical shape, with the plunger knob 908 having a corresponding conical surface. The shapes of both the receptacle 914 and plunger knob 908 may be varied, and need not be limited to conical shapes, nor even to corresponding shapes. Slot 918 is an opening on the outer surface of bent shaft 912, and may be positioned near the distal end of the funnel assembly 910. The slot 918 may provide for an additional aperture through which bone graft material may flow during injection to the treatment site, as described in more detail below.

In use, bone graft material is introduced into the lumen 916 of the funnel assembly 910. The bone graft material may either be introduced through the receptacle 914 at the proximal end, or it may be back-filled by inserting the bone graft material through the opening in the distal end of the funnel assembly 910. Upon insertion of the plunger assembly 900 into the funnel assembly 910, the distal tip 906 pushes the bone graft material along the length of the bent shaft 912 and eventually out of the funnel assembly 910.

It should also be noted that bone chips and/or autograft must be made into pieces small enough to flow through the funnel assembly 910. Otherwise, the funnel assembly 910 may become congested and the bone graft may not flow into the target site as desired.

Once the bone graft material is loaded into the funnel assembly, the bone graft material can be deployed at the target site. The funnel assembly can be inserted into the access cannula until the distal tip of the funnel assembly is positioned adjacent to the target site. The location of the distal tip of the funnel instrument can be modified to any desired location for deploying the graft material at the target site. Due to the bend in the funnel assembly 910, the device may be rotated within the access cannula in order to achieve different angles of approach. The bend may therefore provide for improved access to different regions of the intervertebral disc space. Then, inserting the plunger assembly 900 through the funnel assembly 910, a desired amount of graft material can be injected at the target site. In certain embodiments, the funnel assembly 910 and plunger assembly 900 can each be placed over a k-wire. The plunger assembly 900 can then be advanced into the funnel assembly 910 to deploy the graft into the disc.

As the bone graft material flows through the lumen 916 of funnel assembly 910, it passes slot 918 near the distal end of the bent tube 912. In some embodiments, the opening of slot 918 is smaller than the opening of lumen 916, such that, absent backpressure, bone graft material preferentially exits the funnel assembly 910 through the distal opening of lumen 916. As the target site is filled with bone graft material, however, it may become increasingly difficult to advance the plunger assembly 900 and introduce new bone graft material through the lumen 916. In the event that such resistance is present, some of the bone graft material may be forced through slot 918, thereby providing an alternate distribution route for the bone graft material. In certain embodiments, a plurality of slots 918 may be provided around the circumference of bent shaft 912. The position of slot 918 may be varied depending on the desired distribution of bone graft material at the treatment site. As discussed above, the funnel assembly 910 may be rotated within the access cannula, allowing for bone graft material exiting the slot 918 to be deposited in various locations at the treatment site.

Once the implant and, if applicable, bone graft material have been inserted into the intervertebral disc space, supplemental internal spinal fixation can be employed to facilitate fusion. For example, spinal fixation can include facet screw fixation systems, facet compression devices, and/or posterior pedicle screw and rod systems.

Although the embodiments shown herein depict a dilation introducer with three dilator tubes and one access cannula, other variations are possible. For instance, as noted above, a dilation introducer may include only two dilator tubes and an access cannula. In another embodiment, a dilation introducer may include four or more dilator tubes and an access cannula. In a modified arrangement, the access cannula would be replaced by a dilator tube, wherein the dilator tube with cutting flutes would remain in place, with the inner dilator tubes removed to provide access for surgical tools. The skilled artisan will readily ascertain that many variations of this sort are possible without departing from the scope of the present invention.

The specific dimensions of any of the embodiment disclosed herein can be readily varied depending upon the intended application, as will be apparent to those of skill in the art in view of the disclosure herein. Moreover, although the present inventions have been described in terms of certain preferred embodiments, other embodiments of the inventions including variations in the number of parts, dimensions, configuration and materials will be apparent to those of skill in the art in view of the disclosure herein. In addition, all features discussed in connection with any one embodiment herein can be readily adapted for use in other embodiments herein to form various combinations and sub-combinations. The use of different terms or reference numerals for similar features in different embodiments does not imply differences other than those which may be expressly set forth. Accordingly, the present inventions are intended to be described solely by reference to the appended claims, and not limited to the preferred embodiments disclosed herein.

What is claimed is:

1. A dilation introducer for orthopedic surgery comprising:
a first dilator tube having a substantially circular cross-section;
a second dilator tube having a first longitudinal lumen configured to slidably receive the first dilator therein, wherein the outer surface of the second dilator tube has a substantially rectangular cross-section; and
an access cannula having a second longitudinal lumen configured to slidably receive the second dilator therein, wherein the cross-section of the second longitudinal lumen is substantially rectangular.

2. The dilation introducer of claim 1, wherein the cross-section of the second longitudinal lumen is substantially square.

3. The dilation introducer of claim 2, wherein the second longitudinal lumen has a height and a width of approximately 10 mm.

4. The dilation introducer of claim 1, wherein the cross-section of second longitudinal lumen configured to receive an intervertebral implant therethrough.

5. The dilation introducer of claim 1, wherein the first longitudinal lumen is centered with respect to the outer surface of the second dilator tube.

6. The dilation introducer of claim 1, wherein the access cannula comprises an outer surface having a substantially rectangular cross-section.

7. The dilation introducer of claim 1, wherein a distal end of the access cannula is beveled such that a cross-section of the second longitudinal lumen at the distal end of the access cannula is U-shaped.

8. The dilation introducer of claim 1, configured for removably connecting the first and second dilator tubes together in a locked arrangement, whereby in the locked arrangement the slidable movement is restricted.

9. The dilation introducer of claim 1, whereby the second dilator tube is rotatable with respect to the first dilator tube around the first longitudinal axis.

10. The dilation introducer of claim 1, wherein the first dilator tube contains cutting flutes on at least one side.

11. The dilation introducer of claim 1, wherein the access cannula has a smooth outer surface.

12. A method for accessing a patient's intervertebral disc to be treated in orthopedic surgery, comprising the steps of:
passing a first dilator tube along a first longitudinal axis through Kambin's triangle until the first dilator tube reaches the intervertebral disc to be treated;
passing a second dilator tube along a second longitudinal axis that is parallel to and laterally displaced from the first longitudinal axis, until the distal end of the second dilator contacts the annulus, wherein the second dilator tube has cutting flutes oriented towards the inferior pedicle, and wherein the distal portion of the second dilator tube has a generally semi-annular cross-section, configured such that the second dilator tube does not contact the exiting nerve during insertion;
passing an access cannula over the second dilator tube until the distal end of the access cannula contacts the annulus, wherein the access cannula has an outer surface with a substantially rectangular cross-section.

13. The method of claim 12, further comprising:
passing a third dilator tube over the second dilator tube along the second longitudinal axis until the distal end of the third dilator contacts the annulus, wherein the distal portion of the third dilator tube is beveled such that the third dilator tube does not contact the exiting nerve during insertion,
wherein the access cannula is passed over the third dilator tube.

14. The method of claim 12, further comprising forming a further recess in the inferior pedicle by rotating the second dilator tube back and forth.

15. The method of claim 12, further comprising forming a further recess in the inferior pedicle by longitudinally sliding the second dilator tube back and forth.

16. The method of claim 13, wherein the distal portion of the access cannula has a U-shaped cross-section, the method further comprising:
passing the access cannula over the third dilator tube until the distal end of the third dilator contacts the annulus such that the access cannula does not contact the exiting nerve during insertion;
rotating the access cannula such that generally U-shaped cross-section opens opposite the exiting nerve;
removing the first, second, and third dilator tubes.

17. The method of claim 12, further comprising:
operating on an intervertebral disc by inserting surgical instruments through the access cannula.

18. A method for performing orthopedic surgery, comprising:
enlarging a Kambin's triangle of a patient; and
introducing an access cannula into the Kambin's triangle, the access cannula having a substantially rectangular cross-section.

19. The method of claim 18, further comprising:
removing bone from the inferior pedicle with the first dilator tube prior to introducing the access cannula.

20. The method of claim 18, further comprising operating on the spine through the access cannula.

21. A method for accessing a patient's intervertebral disc to be treated in orthopedic surgery, comprising the steps of:
performing a foraminoplasty;
inserting an access cannula through the enlarged opening created by the foraminoplasty, the access cannula having a substantially rectangular cross-section; and
introducing devices or tools into the intervertebral disc through the access cannula.

22. The method of claim 21, further comprising introducing an implant into the intervertebral disc.

23. The method of claim 22, further comprising expanding the implant within the disc.

24. The method of claim 21, wherein the foraminoplasty is performed at least partially using cutting surfaces on one or more dilator tubes.

25. The method of claim 21, further comprising inserting trans-facet screws into a facet joint.
